(19) United States
(12) Patent Application Publication
(10) Pub. No.: US 2025/0318923 A1
(43) Pub. Date: Oct. 16, 2025

(54) HEART VALVE PROSTHESIS

(71) Applicant: MEDTRONIC, INC., Minneapolis, MN (US)

(72) Inventors: Emily A. Grimm, Petaluma, CA (US); Matthew E. Genovese, Windsor, CA (US); Stuart E. Kari, Windsor, CA (US); Fatemeh Fatemi Far, Santa Rosa, CA (US); Cahal McVeigh, White Bear Township, MN (US); Caitlin Owenson, Santa Rosa, CA (US)

(73) Assignee: MEDTRONIC, INC., Minneapolis, MN (US)

(21) Appl. No.: 18/865,384

(22) PCT Filed: May 11, 2023
(86) PCT No.: PCT/IB2023/054898

§ 371 Date: Nov. 13, 2024

Related U.S. Application Data
(60) Provisional application No. 63/342,926, filed on May 17, 2022.
(60) Provisional application No. 63/406,774, filed on Sep. 15, 2022.
(60) Provisional application No. 63/453,692, filed on Mar. 21, 2023.

(51) Int. Cl. A61F 2/24

(52) U.S. Cl. CPC A61F 2/2418; A61F 2220/0025

(57) ABSTRACT

A heart valve prosthesis includes an inner stent, an outer stent at least partially surrounding the inner stent, and a prosthetic valve operatively coupled to the inner stent. The outer stent includes a plurality of struts and nodes defining open cells of the outer stent, and a plurality of cleats. The plurality of cleats includes first strut cleats extending radially outwardly and proximally from first struts of a first row of the plurality of struts with the heart valve prosthesis in a radially expanded configuration.

### FIELD OF THE INVENTION

The present technology is generally related to prosthetic valves, and in particular transcatheter heart valve prostheses including an inner stent and an outer stent.

### BACKGROUND OF THE INVENTION

The human heart is a four chambered, muscular organ that provides blood circulation through the body during a cardiac cycle. Within the heart there are four valves that control blood flow through the heart's chambers: the mitral valve, the tricuspid valve, the aortic valve, and the pulmonary valve. To ensure blood flows in only one direction, atrioventricular valves (the tricuspid and mitral valves) are present between the junction of the atrium and the ventricles, and semi-lunar valves (pulmonary and aortic valves) govern the exits of the ventricles leading to the lungs and the rest of the body. Each of these valves contain native leaflets that open and close in response to changes in blood pressure as the heart contracts and relaxes. When a valve does not open or close properly, either due to defect or damage, diseases such as stenosis and valvular insufficiency or regurgitation can occur, leading to serious physiological consequences.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Prosthetic heart valves have been developed for repair and replacement of diseased or damage heart valves. The prosthetic heart valve can be compressed or reduced in diameter and can be deployed at the site of the disease heart valve through catheter-based delivery systems. Once the prosthetic valve is positioned at the treatment site, for instance, within a mitral valve, the prosthetic heart valve can be expanded to hold the prosthetic heart valve in place.

While these valve prostheses offer minimally invasive methods for heart valve repair and/or replacement, challenges remain such as reducing a profile of a heart valve prosthesis while maintaining required performance in vivo. A challenge relates to providing a valve prosthesis with a lower profile while maintaining the ability of the valve prosthesis to withstand external forces, maintain function as a valve, and prevent migration while reducing the total amount of material (e.g., metal, fabric, or tissue) present within the mitral valve prosthesis.

### BRIEF SUMMARY OF THE INVENTION

In a first example, a heart valve prosthesis having first and second ends is configured to be radially compressed to a radially compressed configuration and radially expanded to a radially expanded configuration. The heart valve prosthesis includes an inner stent, and outer stent surrounding at least a portion of the inner stent, and a prosthetic valve operatively coupled to the inner stent. The outer stent is operatively coupled to the inner stent and is configured to secure the heart valve prosthesis to native heart tissue. The outer stent includes a plurality of struts and a plurality of nodes defining open cells of the outer stent, and a plurality of cleats. The plurality of cleats include first strut cleats extending radially outwardly and toward the first end of the prosthesis from first struts of a first row of the plurality of struts in the radially expanded configuration.

In a second example, in the heart valve prosthesis according to any of the previous or subsequent examples herein, the plurality of nodes includes a first row of first nodes around a circumference of the outer stent at a first end of the outer stent and a second row of second nodes that is closer to the second end of the heart valve prosthesis then the first row of first nodes, and the first row of first struts have a first end coupled to a corresponding first node and a second end coupled to a corresponding second node.

In a third example, in the heart valve prosthesis according to any of the previous or subsequent examples herein, the plurality of cleats further includes a plurality of second node cleats extending radially outwardly and from corresponding second nodes of the second row of second nodes and in the direction of the first end of the heart valve prosthesis in the radially expanded configuration.

In a fourth example, in the heart valve prosthesis according to any of the previous or subsequent examples herein, the plurality of nodes further includes a third row of third nodes around a circumference of the outer stent that is closer to the second end of the heart valve prosthesis than the second row of second nodes, and the plurality of struts includes a second row of second struts having a first end coupled to a corresponding second node and a second end coupled to a corresponding third node.

In a fifth example, in the heart valve prosthesis according to any of the previous or subsequent examples herein, the plurality of cleats further includes a plurality of third node cleats extending radially outwardly from corresponding third nodes of the third row of third nodes and in the direction of the first end of the heart valve prosthesis in the radially expanded configuration.

In a sixth example, in the heart valve prosthesis according to any of the previous or subsequent examples herein, the third node cleats are forked cleats.

In a seventh example, in the heart valve prosthesis according to any of the previous or subsequent examples herein, the outer stent further includes a plurality of second strut cleats extending radially outwardly from the second struts and in the direction of the first end of the heart valve prosthesis in the radially expanded configuration.

In an eighth example, in the heart valve prosthesis according to any of the previous or subsequent examples herein, the plurality of cleats further includes a plurality of first node cleats extending radially outwardly from corresponding first nodes of the first row of first nodes and in the direction of the first end of the heart valve prosthesis in the radially expanded configuration.

In a ninth example, in the heart valve prosthesis according to any of the previous or subsequent examples herein, the outer stent and the inner stent are operatively coupled to each other at outflow ends thereof.

In a tenth example, in the heart valve prosthesis according to any of the previous or subsequent examples herein, the outer stent and the inner stent are operatively coupled to each other at inflow ends thereof.

In an eleventh example, in the heart valve prosthesis according to any of the previous or subsequent examples herein, the outer stent includes a first end coupled to the inner stent, a transition region flaring radially outwardly from the first end, and a fixation region extending from the transition region to a second end of the outer stent.

In a twelfth example, in the heart valve prosthesis according to any of the previous or subsequent examples herein, the fixation region is concave with respect to a central longitudinal axis of the transcatheter heart valve prosthesis.

In a thirteenth example, in the heart valve prosthesis according to any of the previous or subsequent examples herein, the fixation region is convex with respect to a central longitudinal axis of the transcatheter heart valve prosthesis.

In a fourteenth example, in the heart valve prosthesis according to any of the previous or subsequent examples herein, an outer diameter of the fixation region curves radially outwardly adjacent a junction of the fixation region and the transition region.

In a fifteenth example, in the heart valve prosthesis according to any of the previous or subsequent examples herein, the first end is an inflow end of the inner stent.

In a sixteenth example, in the heart valve prosthesis according to any of the previous or subsequent examples herein, the first end is an outflow end of the inner stent.

In a seventeenth example, in the heart valve prosthesis according to any of the previous or subsequent examples herein, the first strut cleats extend from a portion of the first struts that is between a first node and a second node at respective first and second ends of the first struts.

In an eighteenth example, in the heart valve prosthesis according to any of the previous or subsequent examples herein, the first strut cleats extend from the first struts at or around the midpoint on the first struts and between a first node and a second node at respective first and second ends of the first struts.

In a nineteenth example, in the heart valve prosthesis according to any of the previous or subsequent examples herein, the first strut cleats extend from the first struts at or around the midpoint on the first struts and between a first node and a second node at respective first and second ends of the first struts.

In a twentieth example, in the heart valve prosthesis according to any of the previous or subsequent examples herein, the inner stent and outer stent are operatively coupled where a plurality of inner-stent apertures and a plurality of outer-stent apertures align with one another and wherein the inner stent or the outer stent comprises a connector for coupling the heart valve prosthesis to a delivery system.

In a twenty-first example, a method of treating a native heart valve includes delivering a heart valve prosthesis to a site of the native heart valve and deploying the heart valve prosthesis at the site of the native heart valve. The heart valve prosthesis includes an inner stent, an outer stent surrounding at least a portion of the inner stent and operatively coupled to the inner stent, and a prosthetic valve operatively coupled to the inner stent. The outer stent includes a plurality of struts and a plurality of nodes defining open cells of the outer stent, and a plurality of cleats, the plurality of cleats including first strut cleats coupled to first struts of a first row of the plurality of struts. The heart valve prosthesis is deployed at the site of the native heart valve with the outer stent disposed within leaflets of the native heart valve such that the first strut cleats extend radially outwardly, toward the first end of the heart valve prosthesis, and into tissue surrounding the native heart valve to secure the heart valve prosthesis to the native heart valve.

In a twenty-second example, in the method according to any of the previous or subsequent examples herein: the plurality of nodes includes a first row of first nodes around a circumference of the outer stent at a first end of the outer stent and a second row of second nodes that is closer to the second end of the heart valve prosthesis then the first row of first nodes, and wherein the first row of first struts have a first end coupled to a corresponding first node and a second end coupled to a corresponding second node; the plurality of cleats further includes a plurality of second node cleats extending from corresponding second nodes of the second row of second nodes; and deploying the heart valve prosthesis comprises the plurality of second node cleats extending radially outwardly and towards the first end of the heart valve prosthesis and into the tissue surrounding the native heart valve.

In a twenty-third example, in the method according to any of the previous or subsequent examples herein: the plurality of nodes further includes a third row of third nodes around a circumference of the outer stent that is closer to the second end of the heart valve prosthesis than the second row of second nodes, and wherein the plurality of struts includes a second row of second struts having a first end coupled to a corresponding second node and a second end coupled to a corresponding third node; the plurality of cleats further includes a plurality of third node cleats extending from corresponding third nodes of the third row of third nodes; and deploying the heart valve prosthesis comprises the plurality of third node cleats extending radially outwardly and towards the first end of the heart valve prosthesis and into the tissue surrounding the native heart valve.

In a twenty-fourth example, in the method according to any of the previous or subsequent examples herein, the third node cleats are forked cleats.

In a twenty-fifth example, in the method according to any of the previous or subsequent examples herein: the outer stent further includes a plurality of second strut cleats extending from the second struts; and deploying the heart valve prosthesis comprises the second strut cleats extending radially outwardly and towards the first end of the heart valve prosthesis and into the tissue surrounding the native heart valve.

In a twenty-sixth example, in the method according to any of the previous or subsequent examples herein: the plurality of cleats further includes a plurality of first node cleats extending from corresponding first nodes of the first row of first nodes; and deploying the heart valve prosthesis comprises the plurality of first node cleats extending radially outwardly and towards the first end of the heart valve prosthesis and into the tissue surrounding the native heart valve.

In a twenty-seventh example, in the method according to any of the previous or subsequent examples herein: the inner stent or the outer stent comprises a connector; the connector is coupled to a delivery system during the delivery of the heart valve prosthesis to the site of the native heart valve; and deploying the heart valve prosthesis comprising releasing the connector from the delivery system.

In a twenty-eight example, a heart valve prosthesis comprises an inner stent including a plurality of cells positioned around a central longitudinal axis of the heart valve prosthesis. Each cell is being defined by: two first struts, each proximal strut having a first width; a first crown joining first ends of the first struts; two second struts, each second strut having a second width that is larger than the first width; a second crown joining second ends of the distal struts; and two connectors, each connector coupling a second end of a respective one of the two first struts to a first end of a respective one of the two second struts. The heart valve prosthesis further comprises an outer stent surrounding at least a portion of the inner stent and operatively coupled to the inner stent, and configured to secure the heart valve prosthesis to native heart tissue, and a prosthetic valve operatively coupled to the inner stent.

In a twenty-ninth example, the heart valve prosthesis according to any of the previous or subsequent examples herein further comprises an aperture at the second crown of at least one of the cells.

In a thirtieth example, in the heart valve prosthesis according to any of the previous or subsequent examples herein, the second width of the second struts varies along the length of the second strut.

In a thirty-first example, in the heart valve prosthesis according to any of the previous or subsequent examples herein, at least some of the connectors include at least one connector aperture.

In a thirty-second example, in the heart valve prosthesis according to any of the previous or subsequent examples herein, the at least one connector aperture is configured to operatively attach the prosthetic valve to the connector.

In a thirty-third example, in the heart valve prosthesis according to any of the previous or subsequent examples herein, the inner stent comprises exactly six cells around a circumference of the inner stent.

In a thirty-fourth example, in the heart valve prosthesis according to any of the previous or subsequent examples herein, the inner stent comprises exactly twelve first struts, twelve first crowns, twelve second struts, twelve second crowns, and six connectors.

In a thirty-fifth example, in the heart valve prosthesis according to any of the previous or subsequent examples herein, each of the connectors includes at least one connector aperture.

In a thirty-sixth example, in the heart valve prosthesis according to any of the previous or subsequent examples herein, exactly three of the connector apertures include at least one connector aperture configured to operatively attach the prosthetic valve to the respective connector.

In a thirty-seventh example, in the heart valve prosthesis according to any of the previous or subsequent examples herein, the first struts are proximal struts, the first crown is a proximal crown, the second struts are distal struts, and the second crown is a distal crown, and wherein the outer stent is operatively coupled to the inner stent at an outflow end of the prosthetic heart valve.

In a thirty-eighth example, in the heart valve prosthesis according to any of the previous or subsequent examples herein, the outer stent is operatively coupled to the inner stent at the respective distal crowns of the cells of the inner stent.

In a thirty-ninth example, in the heart valve prosthesis according to any of the previous or subsequent examples herein, the first struts are distal struts, the first crown is a distal crown, the second struts are proximal struts, and the second crown is a proximal crown, and wherein the outer stent is operatively coupled to the inner stent at an inflow end of the prosthetic heart valve.

In a fortieth example, in the heart valve prosthesis according to any of the previous or subsequent examples herein, the outer stent is operatively coupled to the inner stent at the respective proximal crowns of the cells of the inner stent.

In a forty-first example, a heart valve prosthesis comprises an inner stent including a plurality of cells positioned around a central longitudinal axis of the heart valve prosthesis. Each cell is defined by: two first struts; a first crown joining first ends of the first struts; two second struts; a second crown joining second ends of the second struts; and two connectors, each connector coupling a second end of a respective one of the two first struts to a first end of a respective one of the two second struts. The plurality of support struts include: a first support strut having a second end thereof coupled to the second end of a first one of the second struts and/or the second crown of the first second strut; a second support strut having a second end thereof coupled to the second end of a second one of the second struts circumferentially adjacent to the first second strut and/or the second crown of the second support strut; and a support crown joining first ends of the first support strut and the second support strut. The support struts are arranged between circumferentially adjacent second crowns of the inner stent in an inverted V-shaped configuration. The heart valve prosthesis further includes an outer stent at least partially surrounding inner stent and operatively coupled to the inner stent and configured to secure the heart valve prosthesis to native heart tissue, and a prosthetic valve operatively coupled to the inner stent.

In a forty-second example, the heart valve prosthesis according to any of the previous or subsequent examples further comprises an aperture at the second crown of at least one of the cells.

In a forty-third example, in the heart valve prosthesis according to any of the previous or subsequent examples herein, at least some of the connectors include at least one connector aperture.

In a forty-fourth example, in the heart valve prosthesis according to any of the previous or subsequent examples herein, the at least one connector aperture is configured to operatively attach the prosthetic valve to the connector.

In a forty-fifth example, in the heart valve prosthesis according to any of the previous or subsequent examples herein, the inner stent comprises exactly six cells around a circumference of the inner stent.

In a forty-sixth example, in the heart valve prosthesis according to any of the previous or subsequent examples herein, the inner stent comprises exactly twelve proximal struts, twelve proximal crowns, twelve distal struts, twelve distal crowns, six connectors, twelve support struts, and six support crowns.

In a forty-seventh example, in the heart valve prosthesis according to any of the previous or subsequent examples herein, each of the connectors includes at least one connector aperture.

In a forty-eighth example, in the heart valve prosthesis according to any of the previous or subsequent examples herein, exactly three of the connector apertures include at least one connector aperture configured to operatively attach the prosthetic valve to the respective connector.

In a forty-ninth example, in the heart valve prosthesis according to any of the previous or subsequent examples herein, the first struts are proximal struts, the first crown is a proximal crown, the second struts are distal struts, the second crown is a distal crown, the first ends of the first and second support struts are proximal ends, and the second ends of the first and second support struts are distal ends, and wherein the outer stent is operatively coupled to the inner stent at an outflow end of the prosthetic heart valve.

In a fiftieth example, in the heart valve prosthesis according to any of the previous or subsequent examples herein, the outer stent is operatively coupled to the inner stent at the respective distal crowns of the cells of the inner stent.

In a fifty-first example, in the heart valve prosthesis according to any of the previous or subsequent examples herein, the first struts are distal struts, the first crown is a distal crown, the second struts are proximal struts, the second crown is a proximal crown, the first ends of the first and second support struts are distal ends, and the second ends of the first and second support struts are proximal ends, and wherein the outer stent is operatively coupled to the inner stent at an inflow end of the prosthetic heart valve.

In a fifty-second example, in the heart valve prosthesis according to any of the previous or subsequent examples herein, the outer stent is operatively coupled to the inner stent at the respective proximal crowns of the cells of the inner stent.

### BRIEF DESCRIPTION OF DRAWINGS

The foregoing and other features and advantages of the invention will be apparent from the following description of embodiments thereof as illustrated in the accompanying drawings. The accompanying drawings, which are incorporated herein and form a part of the specification, further serve to explain the principles of the invention and to enable a person skilled in the art to make and use the invention. The drawings are not to scale.

FIG. 1 depicts a perspective view of a heart valve prosthesis in accordance with aspects of the disclosure.

FIG. 2 depicts a perspective view of an outflow end of the heart valve prosthesis of FIG. 1 in accordance with aspects of the disclosure.

FIG. 3 depicts a side view of a frame of the heart valve prosthesis of FIG. 1 in accordance with aspects of the disclosure, the frame including an inner stent and an outer stent.

FIG. 4 depicts a top view of an inflow end the frame of FIG. 3 in accordance with aspects of the disclosure.

FIG. 5 depicts a perspective view of the inner stent of the frame of FIG. 3 in accordance with aspects of the disclosure.

FIG. 6 depicts a side view of the inner stent of the frame of FIG. 3 in accordance with aspects of the disclosure.

FIG. 7 depicts an as-cut pattern of a cell of the inner stent of FIG. 5 in accordance with aspects of the disclosure.

FIG. 8 depicts a side view of another embodiment of an inner stent of a frame in accordance with aspects of the disclosure.

FIG. 9 depicts an as-cut pattern of a cell of the inner stent of FIG. 8 in accordance with aspects of the disclosure.

FIG. 10 depicts a schematic view of the heart valve prosthesis of FIG. 1 deployed in a native mitral valve.

FIG. 11 depicts a perspective view of a heart valve prosthesis in accordance with aspects of the disclosure.

FIG. 12 depicts a side view of a frame of the heart valve prosthesis of FIG. 11 in accordance with aspects of the disclosure, the frame including an inner stent and an outer stent.

FIG. 13 depicts a schematic view of the heart valve prosthesis of FIG. 11 deployed in a native mitral valve.

FIGS. 14A-14C various ways to shape set the outer stent of the frame of FIG. 3 in accordance with aspects of the disclosure.

FIGS. 15A-15H depict a portion of an outer stent of the frame transitioning from a delivery configuration to a deployed configuration in accordance with aspects of the disclosure.

FIG. 16A depicts an embodiment of a portion of the outer stent in the deployed configuration prior to shape-setting in accordance with aspects of the disclosure.

FIG. 16B depicts the portion of the outer stent of FIG. 16A after the cleats have been shape-set in accordance with aspects of the disclosure.

FIG. 16C depicts a perspective view of the portion of the outer stent of FIG. 16B.

FIG. 17A depicts an embodiment of a portion of the outer stent in the deployed configuration prior to shape-setting in accordance with aspects of the disclosure.

FIG. 17B depicts the portion of the outer stent of FIG. 17A after the cleats have been shape-set out in accordance with aspects of the disclosure.

FIG. 17C depicts a perspective view of the portion of the outer stent of FIG. 17B.

FIG. 18A depicts an embodiment of a portion of the outer stent in the deployed configuration prior to shape-setting in accordance with aspects of the disclosure.

FIG. 18B depicts the portion of the outer stent of FIG. 18A after the cleats have been shape-set in accordance with aspects of the disclosure.

FIG. 18C depicts a perspective view of the portion of the outer stent of FIG. 18B.

### DETAILED DESCRIPTION OF THE INVENTION

Specific embodiments of the present invention are now described with reference to the figures, wherein like reference numbers indicate identical or functionally similar elements. The terms “proximal” and “distal”, when used in the following description to refer to the heart valve prosthesis or elements of the heart valve prosthesis are with reference to the direction of the direction of blood flow. Thus, “proximal” refers to positions in an upstream direction with respect to the blood flow and “distal” refers to positions in a downstream direction with respect to blood flow.

The following detailed description is merely exemplary in nature and is not intended to limit the invention or the application and use of the invention. Although the description of the invention is in the context of the treatment of a mitral valve, the invention may be used where it is deemed useful in other anatomical sites. For example, the present invention is not limited to mitral heart valves and may be applied in other locations located within the body, for example, other heart valves and/or venous valves. Furthermore, there is no intention to be bound by any expressed or implied theory presented in the preceding technical field, background, brief summary, or the following detailed description.

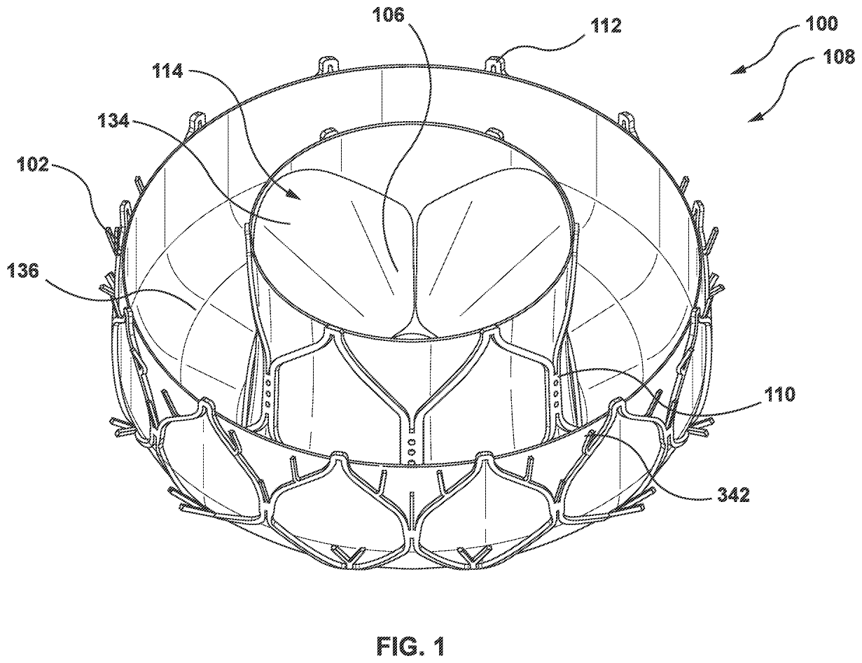

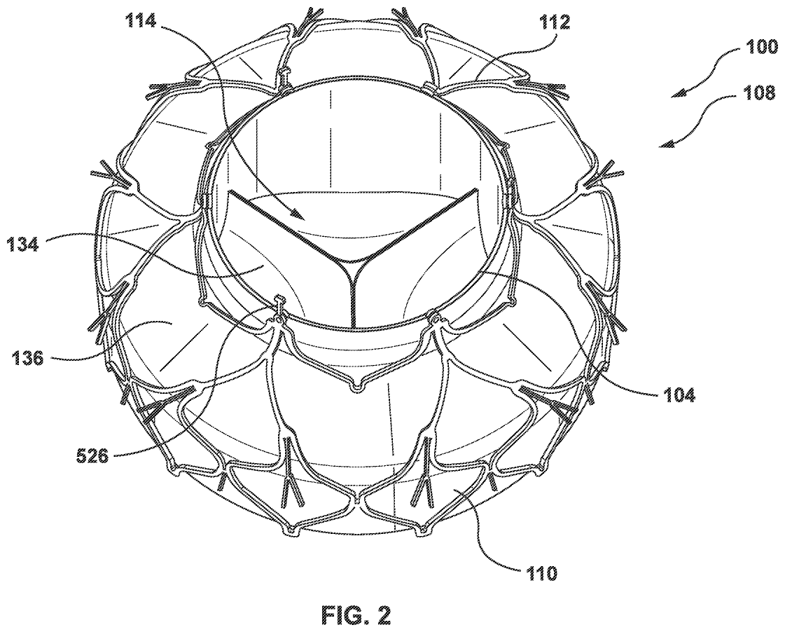

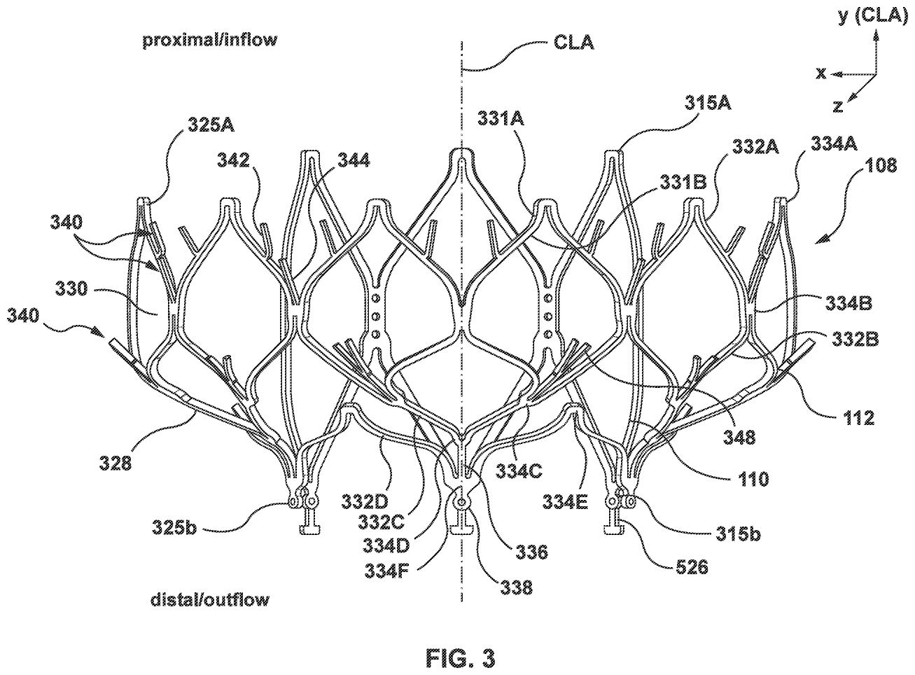

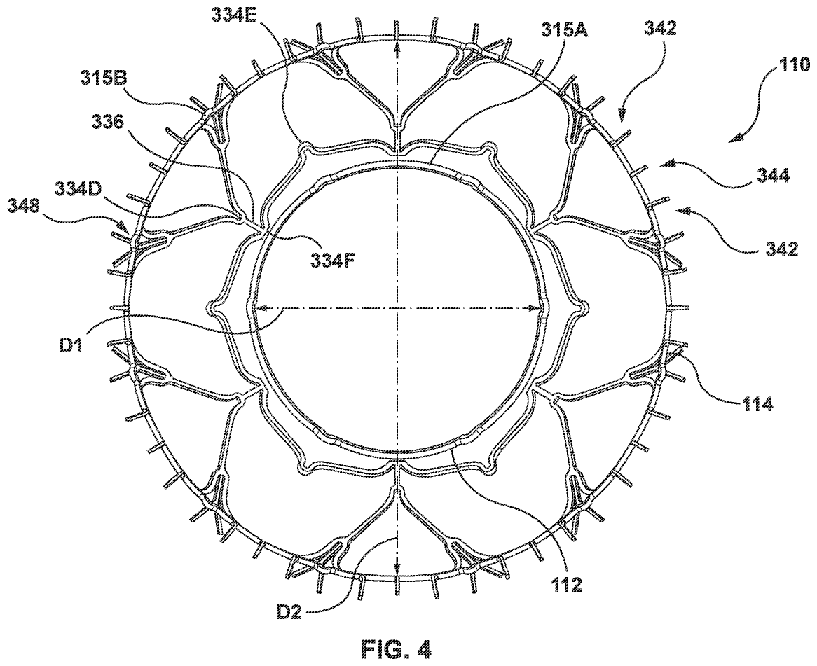

A perspective view of a transcatheter heart valve prosthesis 100 in accordance with an aspect of the disclosure is shown in FIG. 1, with FIG. 2 depicting a distal or outflow view of the heart valve prosthesis 100. The heart valve prosthesis 100 is configured to be compressed into a reduced-diameter delivery configuration within a delivery catheter and to return to an expanded, deployed configuration when delivered and/or released from the delivery catheter within a native mitral valve. The heart valve prosthesis 100 includes a frame 108 and a prosthetic valve 114. The frame 108 is shown in a side view in FIG. 3, with FIG. 4 depicting a proximal or inflow view of the frame 108. The frame 108 has a stent-like structure that is configured to support the prosthetic valve 114 and to define, along a central longitudinal axis CLA thereof, a blood flow lumen that substantially extends from an inflow end 102 to an outflow end 104 of the heart valve prosthesis 100. In the embodiments shown herein, the frame 108 generally includes an inner stent 110 and an outer stent 112. In aspects hereof, the inner stent 110 of the frame 108 may be alternatively referred to as a valve support, an inner frame, and/or a valve housing. Similarly, the outer stent 112 of the frame 108 may alternatively referred to as an anchoring frame, an anchoring member, a fixation ring, and/or an outer frame. The inner stent 110 is configured to hold the prosthetic valve 114, and the outer stent 112, which surrounds the inner stent 110, is configured to secure the heart valve prosthesis 100 to the native tissue of the heart when implanted in vivo. The frame 108 may be considered to have a dual-stent structure, i.e., an inner stent and an outer stent.

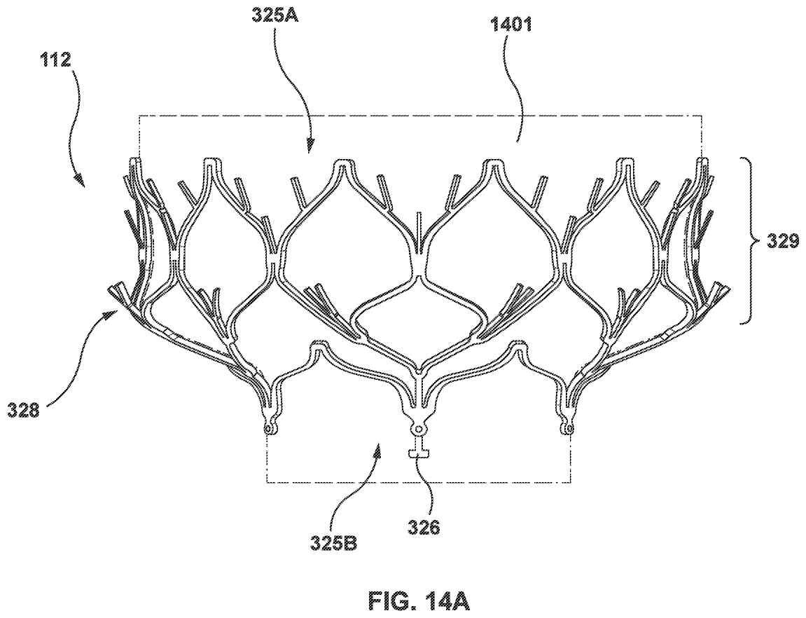

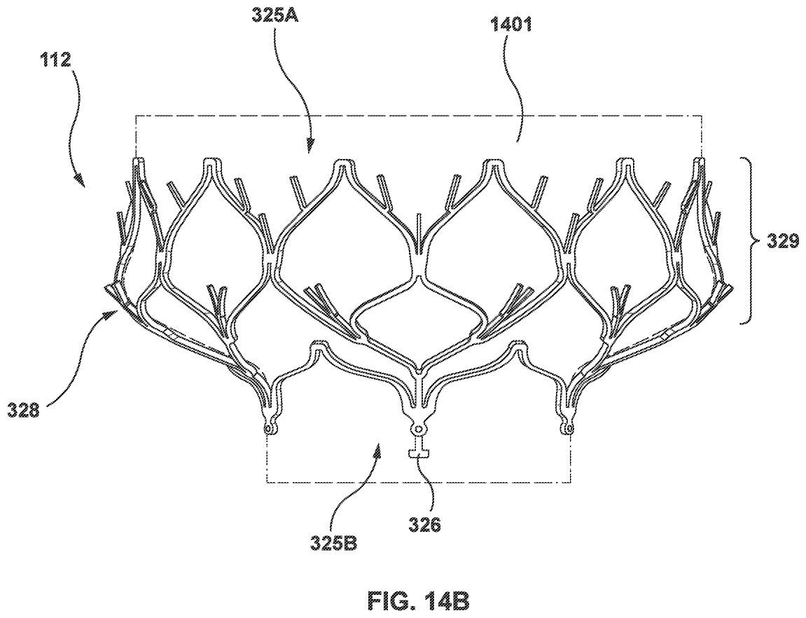

The outer stent 112 is configured to secure the heart valve prosthesis 100 to the native valve and the surrounding tissue, such as the inward facing-surface of the native leaflets. The outer stent 112 is best shown in FIGS. 3 and 4. The outer stent 112 is positioned around the inner stent 110 and defines an inflow or distal end 325A having a first diameter D1 and an outflow or proximal end 325B having a second diameter D2 that is smaller than the first diameter D1. A transition portion 328 is positioned between the inflow end 325A and the outflow end 325B of the outer stent 112. As shown in FIG. 3, the transition portion 328 reduces in diameter between the inflow end 325A and the outflow end 325B, acting as a taper between the first diameter DI and the second, smaller diameter D2. In other embodiments, depending on the location of the heart valve prosthesis, the outflow end and the inflow end may be reversed, and/or alternatively referred to as a first end or a second end, without departing from the scope hereof. The diameter of the outer stent 112 may vary from the inflow end 325A to the transition portion 328 of the outer stent 112 based on how the outer frame 112 is shape-set on a mandrel, which will be discussed in further detail below with regard to FIGS. 14A-14C.

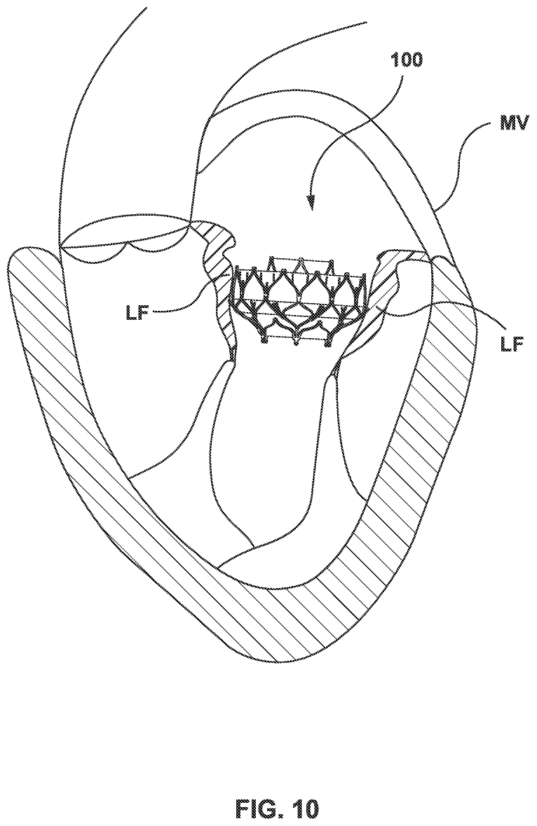

At least a portion of an outer surface of the outer stent 112, when the heart valve prosthesis 100 is in an expanded state, is configured to be disposed against the native tissue of the heart for securing the outer stent 112 and, concurrently, the heart valve prosthesis 100, at the native heart valve, as shown in FIG. 10. Further, a portion of the outer stent 112, in this embodiment the inflow portion, is mechanically isolated from the inner stent 110. In particular, the outer stent 112 may deform upon implantation within a native mitral valve annulus, and/or expand and contract in response to movement of the native tissue, while remaining spaced from the inner stent 110, thereby enabling the inner stent 110 to remain relatively still and undeformed. Therefore, the inner stent 110 is isolated from external forces, In particular, the external forces are absorbed by the outer stent 112 and at the connection between the outer stent 112 and the inner stent 110. Therefore, the external forces are spaced from the portion of the inner stent 110 housing the prosthetic valve 114, thereby enabling the prosthetic valve 114 disposed within the inner stent 110 to more efficiently replicate the function of the native mitral valve. In addition, the outer stent 112 may further include a plurality of prongs or barbs or cleats that extend radially from the outer surface of the outer stent 112 and are configured to engage with the native tissue, further fixating the outer stent 112 to the tissue, which will be explained in further detail below.

As shown in FIG. 3, the outer stent 112 defines a plurality of open cells 330 arranged in a repeating pattern around a circumference of the outer stent 112. In the embodiment shown in FIGS. 3-4, the outer stent 112 includes a proximal or first row of twelve (12) cells 330 around a circumference of the outer stent 112. The outer stent 112 includes additional rows of cells 330 as will be described below. Each cell 330 of the outer stent 112 is defined by a plurality of struts and nodes or crowns connecting adjacent struts. Starting at the inflow end 325A, the outer stent 112 includes a first or proximal row of first or proximal struts 332A connected to each other at first nodes 334A. A second row of second struts 332B are coupled to each other, and to the first struts 332A, at second nodes 334B. A third row of third struts 332C are coupled to each other the second row of struts 332B at third nodes 334C. However, the third struts 332C are not coupled to each other at the third nodes 334C. Fourth nodes 334D couple outflow ends of adjacent pairs of the third struts 332C to each other. Connector arms 336 extend distally and substantially longitudinally from the fourth nodes 334D to sixth nodes 334F. A row of fourth struts 332D extend proximally and circumferentially from the sixth nodes 334F. Proximal ends of adjacent fourth struts 332D are coupled to each other at fifth nodes 334E. The fifth nodes 334E are not attached to struts proximal of the fifth nodes 334E. The inflow end 325A of the outer stent 110 includes exactly twelve first nodes 334A and the outflow end 325B of the outer stent includes exactly six sixth nodes 334F. Extending distally form the sixth nodes 334F are eyelets 338. The eyelets 338 may be used for coupling the outer stent 112 to the inner stent 110 using, for example, sutures, rivets, or hooks. In particular examples, the eyelets 338 are aligned with eyelets 520 of the inner stent 110 and coupled to each other, such as by using a rivet. The description of the struts, cells, and nodes of the outer stent 112 above is merely an example, and is not meant to be limiting. Other arrangements may also be utilized.

As stated previously, the outer stent 112 includes a plurality of cleats 340 that extend radially outwards from the outer surface of the outer stent 112 and are configured to engage with the native tissue. In the embodiment shown in FIGS. 3-4, the outer stent 112 includes a row of proximal strut cleats 342, a row of second node cleats 344, and a row of forked or third node cleats 348. Each proximal strut cleat 342 extends from each proximal strut 332A of the outer stent 112. More specifically, each proximal strut 332A includes a first side 331A that faces the inflow end 325A of the outer stent 112 when the outer stent 112 is in the radially expanded deployed configuration and a second side 331B that opposes the first side 331A and faces the outflow end 325B of the outer stent 112 when the outer stent 112 is in the radially expanded deployed configuration. Each proximal strut cleat 342 is coupled to the first side 331A of each proximal strut 332A of the outer stent 112. The proximal strut cleats 342 may also be referred to as mid-strut cleats as they extend from struts between adjacent nodes. Each second node cleat 344 extends from each second node 334B of the outer stent 112. Each forked cleat 348 extends from each third node 334C of the outer stent 112. Each forked cleat 348 has a substantially “Y-shaped” configuration such that the forked cleat 348 extends from the third node 334C and then forks such that the cleat 348 splits into two cleat ends in a “Y-shaped” configuration. The second node cleats 344 and the proximal strut cleats 342 are substantially straight segments that extend from the second nodes 334B and the proximal struts 332A, respectively.

As best shown in FIGS. 3 and 4, each cleat extends substantially proximally (i.e., towards the inflow end 325A of the outer stent 112) with respect to the central longitudinal axis CLA (y-axis) and radially outwardly (i.e., in the z-axis direction). In other words, in this embodiment, each cleat of the plurality of cleats does not extend in a direction towards the outflow end 325B of the outer stent 112, nor do the cleats extend inwardly towards the central longitudinal axis CLA of the frame 108. As shown, each proximal strut cleat 342 is coupled to a corresponding proximal or first strut 332A and extends proximally with respect to the y-axis and outwardly with respect to the z-axis, i.e., radially outwardly. Similarly, each second node cleat 344 of a corresponding second node 334B and extends proximal with respect to the y-axis and outwardly with respect to the z-axis, i.e., radially outwardly. Similarly, each forked cleat 348 is coupled a corresponding third node 334C and extends proximally with respect to the y-axis and outwardly with respect to the z-axis, i.e., radially outwardly. Thus, each of the cleats 340 extends proximally and radially outwardly.

With respect to the x-axis (i.e., lateral axis), each second node cleat 344 extends in a direction including a vector that is substantially parallel to the CLA of the frame 108. In other words, each second node cleat 344 does not extend primarily laterally or primarily circumferentially. However, those skilled in the art will recognize that stents do not necessary expand uniformly. Therefore, in the radially expanded deployed configuration, the second node cleats 344 may be angled somewhat laterally or circumferentially (i.e., in the x-axis direction) with respect to the central longitudinal axis CLA (i.e., with respect to the y-axis). In some embodiments, this angle may be under 10°.

In addition to extending proximally and radially outwardly, each proximal strut cleat 342 extends at an angle from the proximal strut 332A to which it is coupled. More particularly, each proximal strut cleat 342 extends at an angle in the range of 15°-45°, or 20°-40°, or approximately 30° from the proximal strut 332A to which it is coupled such that the proximal strut cleat 342 is not parallel to the proximal strut 332A to which it is coupled, but rather extends from the proximal strut 332A at an acute angle with respect to the proximal strut 332A.

In addition to extending proximally and radially outwardly, each forked cleat 348 extends from its respective third node 334C at an angle laterally or circumferentially, i.e., in the x-axis direction. More specifically, each forked cleat 348 is disposed between two adjacent second struts 332B. When the outer stent 112 radially expands, one of the two adjacent second struts 332B substantially curves more than the other of the two adjacent second struts 332B, as shown in FIG. 3. This curvature angles the forked cleat 348 towards the straighter of the two adjacent second struts 332B, as shown in FIG. 3. Accordingly, as shown in FIG. 3, every other forked strut 348 extends at an angle to the left (when the outer stent 112 is viewed from the side), and every other forked strut 348 extends at an angle to the right (when the outer stent 112 is viewed from the side). Each forked strut 348 extends at an angle of about 40°-60° laterally with respect to central longitudinal axis CLA.

The proximal strut cleats 342 may have a length of about 2 mm-4 mm, the first node cleats 344 may have a length of about 2 mm-4 mm, and the forked cleats 348 may have a length of about 3 mm-6 mm.

The cleats 340 are shape set to the radially expanded configuration. A tool may be used to move the cleats 340 to the radially expanded configuration prior to shape setting the outer stent 112 to the radially expanded configuration, as shown and described in further detail below.

Those skilled in the art will recognize that cleats 340 may be varied from the embodiment described while remaining in the spirit of this invention. Some other embodiments will be described below to further illustrate. However, these examples are not meant to be limiting, and other embodiments may be utilized in keeping with the scope of the present disclosure.

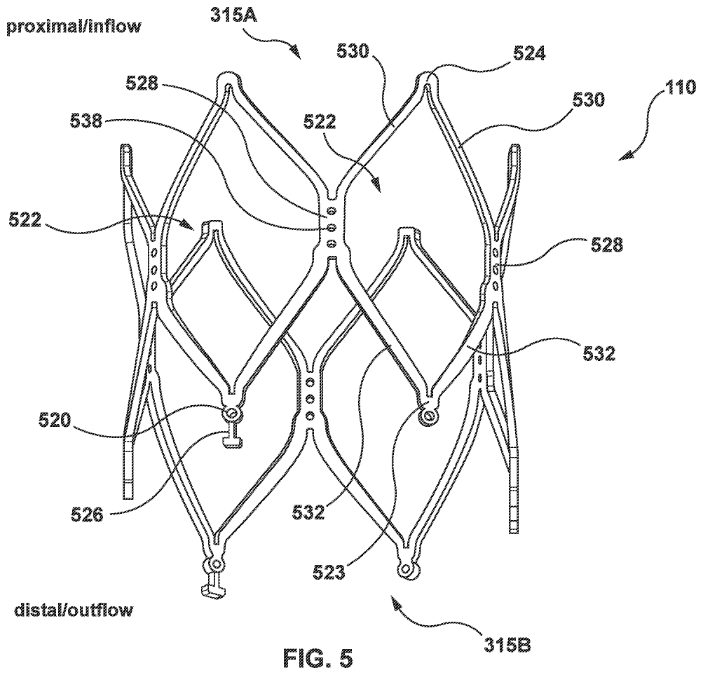

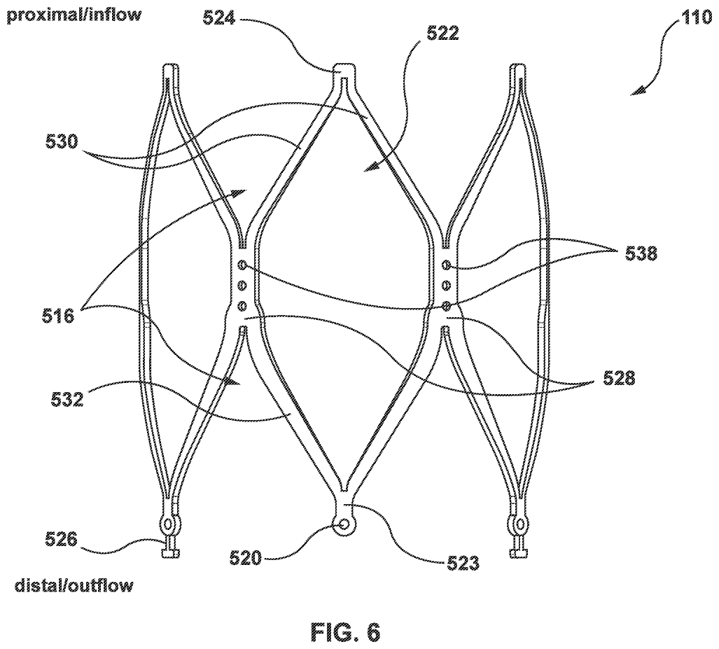

The inner stent 110 is positioned within the outer stent 112 such that at least a portion thereof is spaced from the outer stent 112. FIG. 5 depicts a perspective view of an inner stent 110 of the heart valve prosthesis 100 in accordance with an aspect of this disclosure, while FIG. 6 depicts a side view of the inner stent 110. The inner stent 110 generally forms a hollow cylindrical shape having a substantially constant diameter from an inflow end 315A to an outflow end 315B thereof. The stent-like structure of the inner stent 110 defines a plurality of open cells 522 arranged in a repeating pattern around a circumference of the inner stent 110. In the embodiment of FIGS. 5-7, the inner stent 110 includes six (6) cells around a circumference of the inner stent 110. As would be understood by those skilled in the art, for a given circumference, having fewer cells around the circumference generally leads to less material for the inner stent, which may improve packing density. Thus, an inner stent with six (6) cells around a circumference of the inner stent likely has a lower packing density than an inner stent with twelve cells (12) around the circumference. For example, and not by way of limitation, the heart valve prosthesis 100 with an inner stent 110 having six (6) cells may be delivered in a 24 Fr catheter, whereas a similar heart valve prosthesis including an inner stent having twelve (12) cells may require a 35 Fr catheter.

Each cell 522 of the inner stent 110 is defined by a pair of proximal struts 530, a proximal crown 524 joining the pair of proximal struts 530, a pair of distal struts 532, a distal crown 523 joining the pair of distal struts 530, and a pair of connectors 528, one of the connectors 528 connecting one of the proximal struts 530 to one of the distal struts 532 defining the cell 522 and the other connector 528 connecting the other proximal strut 530 to the other distal strut 532 defining the cell 522. The connectors 528 also connect circumferentially adjacent cells 522 to each other. In the embodiment of FIGS. 5-7, the distal crowns 523 include an aperture 520 or eyelet that enables the inner stent 110 to be coupled to the outer stent 112 using pins, rivets, or other securement devices known in the art. Thus, in the embodiment shown, the inner stent 110 includes six (6) proximal crowns 524, with each proximal crown 524 joining two (2) adjacent proximal struts 530 such that the inner stent 110 includes twelve (12) proximal struts 530. Further, the inner stent 110 includes six (6) distal crowns 523, with each distal crown 5234 joining two (2) adjacent distal struts 532 such that the inner stent 110 includes twelve (12) distal struts 532. Further, the inner stent 110 includes six (6) connectors 528, with each connector 528 connecting two (2) proximal struts 530 of adjacent cells 522 to two (2) distal struts 532 of the adjacent cells 522. Therefore, the inner stent includes six (6) connectors 528 around the circumference of the inner stent 110. Further, in the embodiment of FIGS. 5-7, every other distal crown 523 aperture 520 of the inner stent 110 includes a T-bar connector 526 that extends distally therefrom and that may be used to connect the prosthesis to a delivery system for delivering the prosthesis into a patient's body. Thus, the inner stent 526 includes three T-bar connectors 526. The T-bar connectors 526 of the inner stent 110 enable the inner stent 110 to be coupled or connected to a delivery system. However, this is not meant to be limiting, and there may be more or fewer T-bar connectors 526. The T-bar connectors 526 shown in FIG. 5-7 are T-shaped, but such connectors may be shapes other than T-shaped, such as L-shaped as known to those skilled in the art, as long as the shape is capable of functioning as a connector. Further, instead of or in addition to the inner stent 110 including connectors 526, the outer stent 112 may include T-bar connectors 326 that enable the outer stent 112 to be coupled or connected to a delivery system, which is shown and described with respect to FIGS. 14A-14C below. As would be understood by those skilled in the art, because the outer stent 112 and the inner stent 110 are connected, connecting one to the delivery device connects both to the delivery device.

The inner stent 110 may also be described as including a proximal section including the proximal struts 530 connected at proximal ends thereof by the proximal crowns 524 and at distal ends thereof by the connectors 528, a distal section including the distal struts 532 connected at distal ends thereof by the distal crowns 523 and at proximal ends thereof by the connectors 528, and a central portion comprising the connectors 528 connecting the proximal section to the distal section. The connectors 528 are generally axially oriented, i.e., substantially parallel to the central longitudinal axis of the inner stent 110. The connectors 528 may also act as commissure posts for attaching the prosthetic valve 114 to the inner stent 110. Thus, the connectors 528 may include suture holes or apertures 538 to aid in attaching the prosthetic valve 114 to the inner stent 110. Although all of the connectors 528 are shown with apertures 538, in other embodiments, only connectors 528 that are used as commissure posts have the apertures 538. Thus, for example and not by way of limitation, if the prosthetic valve 114 has three leaflets (and thus three commissures), then three of the connectors 528 may include the apertures 538, such as every other connector 528. In other embodiments, such as the one shown, all of the connectors 528 may include apertures 538 that can be used for connecting the commissures of the prosthetic valve 114 and/or a skirt coupled to the inner stent 110 and/or or not used at all.

As noted above, using a smaller amount of material to form the inner stent 110, such as by limiting the number of cells 522 or struts, or by varying the dimensions, such as width, along individual struts, may provide a benefit of lower packing density. However, the inner stent 110 must still be able to withstand forces on it when deployed at the treatment site, such as a native mitral valve. A particular area of interest for potential forces on the inner stent 110 is the location of the connection between the outer stent 112 and the inner stent 110, that is, the distal crowns 523, as described above.

Accordingly, in the embodiment of FIGS. 5-7, the distal struts 532 have a larger width than the proximal struts 530, resulting in an inner stent 110 that is asymmetric with respect to a lateral axis, that is, an axis perpendicular to the central longitudinal axis of the inner stent 110. Having distal struts 532 with a larger width than the proximal struts 530 provides increased strength at the location of the connection between the inner stent 110 and the outer stent 112, while maintaining a low packing density by having narrower proximal struts 530.

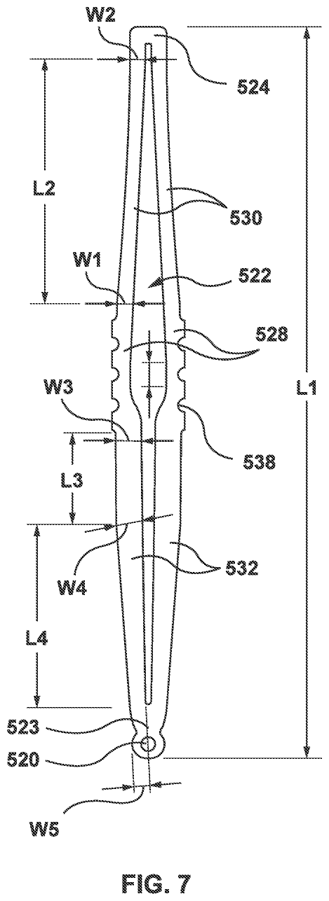

In one exemplary embodiment, shown in FIG. 7, when in the as-cut configuration, each of the cells 522 has a length L1 measured from the proximal end of the proximal crown 524 to the distal end of the distal crown 523 of about 30.34 mm. The proximal struts 530 have a length L2 of approximately 10.15 mm long. Further, the proximal struts 530 have a width W1 at a distal end thereof (adjacent the connectors 528) of about 0.72 mm and a width W2 at proximal end thereof (adjacent the crowns 524) of about 0.65 mm. Thus, the proximal struts 530 taper in width in a proximal direction. As can be seen, the widths at the proximal and distal ends of the proximal struts 530 are similar, although not identical. In the embodiment shown in FIG. 7, the distal struts 532 are wider than the proximal struts 530. In particular, the distal struts 532 have a width W3 at a proximal end thereof (adjacent a distal end of the connectors 528) of about 1.12 mm. Further, at a distance L3 of about 3.76 mm from the distal end of the connectors 528, a width W4 of the distal struts is about 1.11 mm. Thus, this portion of the distal struts 532 is nearly uniform in width. From the distance L2 from the distal end of the connectors 528 to the distal end of the distal struts (adjacent the distal crowns 523), the width of the distal struts tapers from the width W4 of 1.11 mm to a width W5 of about 0.70 mm. Although particular dimensions have been listed, these are merely exemplary, and other dimensions may be utilized depending on several factors, including the packing profile of the heart valve prosthesis, the intended location of the heart valve prosthesis, and other factors know to those skilled in the art. In embodiments, depending on the number of cells, diameter, laser clearance, number of struts, and other factors, the distal struts 532 have a width in the range of about 0.2 mm to about 1.7 mm. Further, the thickness of the struts (radial dimension) may be selected depending on the situation, and the struts may be uniform in thickness or the thickness may vary within a particular strut. In embodiments, the distal struts 532 may be thicker than the proximal struts 530. Further, in embodiments, the distal struts 532 may have a thickness of about 0.5 mm to about 1.0 mm.

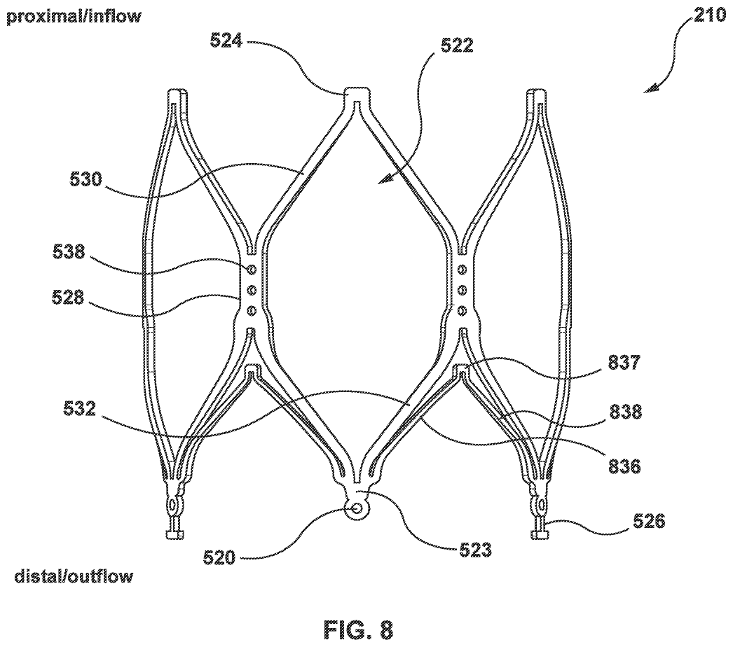

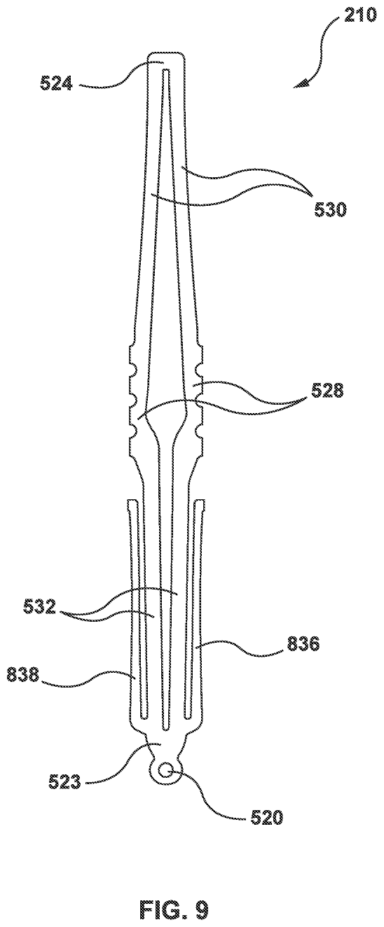

FIGS. 8-9 show another embodiment of an inner stent 210. The inner stent 210 can be used with the outer stent 112, prosthetic valve 114, and other components described above to form the heart valve prosthesis 100 with the inner stent 210 instead of the inner stent 110. Accordingly, all of the descriptions above regarding the outer stent 112, the prosthetic valve 114, skirts, connections between the outer stent 112 and the inner stent 110 are incorporated into this embodiment.

The inner stent 210 of FIGS. 8-9 is similar to the inner stent 110 of FIGS. 5-7 in that the inner stent 210 includes cells 522, proximal struts 530, proximal crowns 524 coupling proximal ends of adjacent proximal struts 530, distal struts 532, distal crowns 523 coupling distal ends of adjacent distal struts 532, and connectors 528 connecting the proximal struts 530 to the distal struts 532 and T-bar connectors 526 extending distally from every other distal crown 523 or aperture 520. As noted above, there may be more or fewer connectors 526, and/or the connectors may be shapes other than T-shaped. The inner stent 210 differs from the inner stent 110 in that the inner stent 210 does not include wider distal struts 532 and the inner stent 210 includes support struts described below. In particular, as shown in FIGS. 8 and 9, a first support strut 836 includes a distal end coupled to and extending from a distal end of a first one of the distal struts 532. The distal end of the first support strut 836 could also be described as coupled to the distal crown 523 at the distal end of the first distal strut 532. The first support strut 836 extends from the distal end of the first distal strut 532 angularly towards the connector 528 at a proximal end of the first distal strut 532. Similarly, a second support strut 838 includes a distal end coupled to and extending from a distal end of a second one of the distal struts 532 that is circumferentially adjacent to the first distal strut 532 (or a circumferentially adjacent distal crown 523) angularly towards the connector 528 shared by the first and second distal struts 532. A support crown 837 connects proximal ends of the first and second support struts 836, 838. In particular, the angular direction of each of the support struts 836, 838 is in a proximal and circumferential direction. Stated another way, the support struts 836, 838 generally follow the direction of the distal struts 532 to which they are attached. Stated another way, the support struts 836, 838 and support crown 837 form a chevron shape or an inverted V-shape between each of the distal crowns 523, with the tip of the inverted V-shape in the proximal direction. FIG. 8 shows the inner stent 210 in a radially expanded configuration such that the angle of the support struts is different than in the as-cut configuration shown in FIG. 9. Further, one set of a first support strut 836, a second support strut 838, and a support crown 837 has been described between circumferentially adjacent distal crowns 523. In the embodiment of FIGS. 8-9, a set of a first support strut 836, a second support strut 838, and a support crown 837 is disposed between each adjacent distal crowns 523, as best seen in FIG. 8. Therefore, as there are six (6) distal crowns 523, there are six (6) sets of first support struts 836, second support struts 838, and support crowns 837.

In the embodiment of FIGS. 8-9, the support struts 836, 838 and support crowns 837 of the inner stent 210 function similarly to the wider distal struts 532 of the inner stent 110 to provides increased strength at the location of the connection between the inner stent 210 and the outer stent 112, while maintaining a low packing density by not adding material to the proximal struts 530.

Those skilled in the art would recognize that although the inner stents 110 and 210 have been described separately with the inner stent 110 including widened distal struts 532 and the inner stent 210 including the support struts 836, 838, the two embodiments could be combined. For example, and not by way of limitation, an inner stent could have widened distal struts 532 of the inner stent 110 and the support struts 836, 838 of the inner stent 210.

In accordance with aspects hereof, the outer stent 112 and the inner stent 110, 210 of the frame 108 of the heart valve prosthesis 100 may be made from any number of suitable biocompatible materials, e.g., stainless steel, nickel titanium alloys such as Nitinol™, cobalt chromium alloys such as MP35N, other alloys such as ELGILOY® (Elgin, Ill.), various polymers, pyrolytic carbon, silicone, polytetrafluoroethylene (PTFE), or any number of other materials or combination of materials. A suitable biocompatible material for the outer stent 112 and the inner stent 110, 210 would be selected to enable the heart valve prosthesis 100 to be compressed into a reduced diameter configuration for transcatheter delivery to a native valve, whereby release from a delivery catheter allows the outer stent 112 and the inner stent 110, 210, and hence the heart valve prosthesis 100, to self-expand, returning to an expanded, deployed configuration. In some embodiments, the self-expansion is accomplished through the use of a shape-memory material such as Nitinol™. The inner stent 110, 210 and the outer stent 110, 112 of the heart valve prosthesis 100 may be processed to have a default or “set” shape that coincides with the deployed configuration. Therefore, once the compressed heart valve prosthesis 100 is delivered and released, the prosthesis 100 will return to the default or “set” deployed configuration. The outer stent 112 and the inner stent 110, 210 may be the same material or may be different materials.

As introduced above, the prosthetic valve 114 of the heart valve prosthesis 100 is capable of regulating flow through the inner stent 110, 210 via valve leaflets 134. FIGS. 1-2 illustrate an exemplary prosthetic valve 114 having three leaflets 134, although a bicuspid leaflet configuration may alternatively be used in embodiments hereof. When deployed in situ, the prosthetic valve 114 in a closed state is configured to block blood flow in one direction to regulate blood flow through the blood flow lumen 106 of the inner stent 110, 210. The valve leaflets 134 are disposed to coapt within the inner stent 110, 210 and are secured to the inner surface of the inner stent 110, such that the valve leaflets 134 open during diastole. Adjoining pairs of leaflets 134 may be attached to one another at their lateral ends to form leaflet commissures. The leaflet commissures in turn may be attached to the inner stent 110, 210 at the connectors 528. The orientation of the leaflets 134 within the inner stent 110, 210 depends upon which end of the heart valve prosthesis 100 is the inflow end 102 and which end of the heart valve prosthesis 100 is the outflow end 104, thereby ensuring one-way flow of blood through the heart valve prosthesis 100.

The leaflets 134 may be formed of various flexible materials including, but not limited to, natural pericardial material such as tissue from bovine, equine or porcine origins, or synthetic materials such as polytetrafluoroethylene (PTFE), DACRON® polyester, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject failure due to stretching, deforming, or fatigue.

In some embodiments, the heart valve prosthesis 100 may further include a skirt 136 that is coupled to the inner stent 110 and/or the stent portion 112. The skirt 136 acts as a seal around the heart valve prosthesis 100 to limit potential paravalvular leaks. The skirt 136 may be a natural or biological material such as pericardium or another membranous tissue such as intestinal submucosa. Alternatively, the skirt 136 may be a low-porosity woven fabric, such as polyester, Dacron fabric or PTFE, which creates a one-way fluid passage when attached to the prosthetic heart valve 100. In one embodiment, the skirt 136 may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side.

The heart valve prosthesis 100 may further include a brim or preshaped wire element that extends outwardly from the inflow end 325A of the outer stent 112. The brim may act as an atrial retainer, if present, and to serve such a function the brim may be configured to engage tissue above a native annulus, such as a supra-annular surface or some other tissue in the left atrium, to thereby inhibit downstream migration of the heart valve prosthesis 100 as well as mitigate any leakage through any gaps between native tissue and the brim, for e.g., during atrial systole. The brim may also serve as a transesophageal echocardiographic landmark that helps position the heart valve prosthesis during implantation.

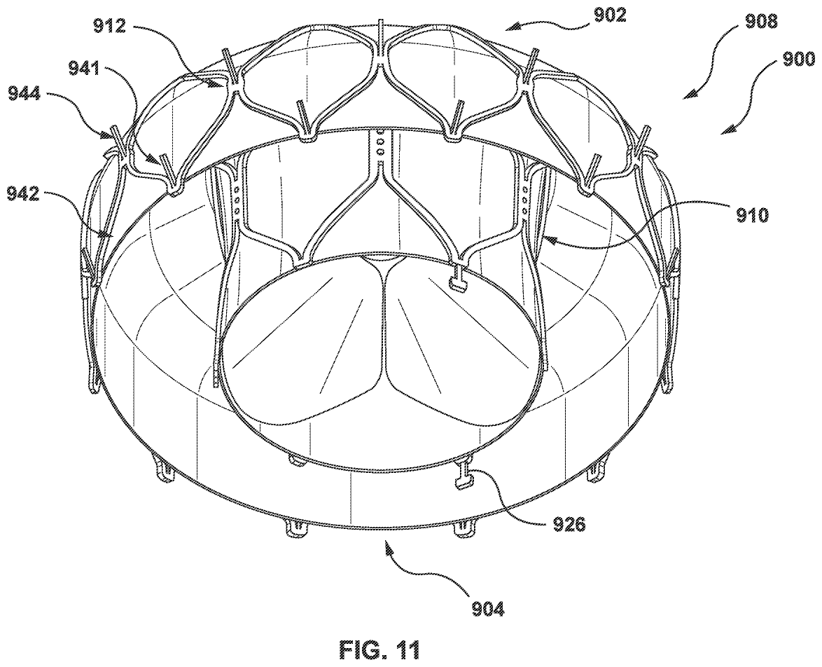

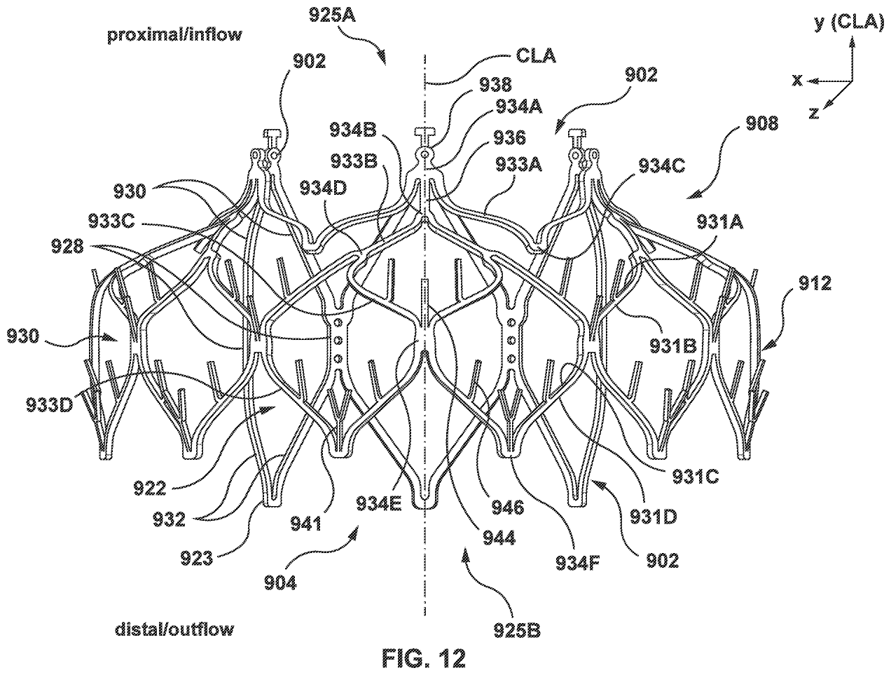

FIGS. 11 and 12 show a perspective view of another heart valve prosthesis 900 in accordance with aspects of the present disclosure. The heart valve prosthesis 900 is similar to the heart valve prosthesis 100 in that it includes a frame 908 including an inner stent 910 and an outer stent 912, and a prosthetic valve 914 disposed within and attached to the inner stent 910. FIGS. 11 and 12 differ from the heart valve prosthesis 100 in that the outer stent 912 and the inner stent 910 are attached to each other at an inflow end 902 of the heart valve prosthesis 900 instead of the outflow end 904, as in the heart valve prosthesis 100.

As such, because the outer stent 912 and the inner stent 910 are attached to each other at the inflow end 902 of the heart valve prosthesis 900, the cells 922 of the heart valve prosthesis inner stent 910 are oriented opposite the cells 522 of the inner stent 110. Thus, similar to the cells 522 of the inner stent 110, each cell 922 of the inner stent 910 is defined by a pair of proximal struts 930, a proximal crown 924 joining the pair of proximal struts 930, a pair of distal struts 932, a distal crown 923 joining the pair of distal struts 930, and a pair of connectors 928, one of the connectors 928 connecting one of the proximal struts 930 to one of the distal struts 932 defining the cell 922 and the other connector 928 connecting the other proximal strut 930 to the other distal strut 932 defining the cell 922. The connectors 928 also connect circumferentially adjacent cells 922 to each other. Similar to the inner stent 110 described above, the inner stent 910 includes T-bar connectors 926 (which may take on other shapes, such as L-shaped connectors, as also described above) that couple the inner stent 110 to a delivery system. The T-bar connectors 926 of the inner stent 910 extend distally from every other distal crown 923 of the inner stent 910, as shown in FIG. 12. In the embodiment of FIGS. 11-12, the proximal crowns 924 include an aperture 920 that enables the inner stent 910 to be coupled to the outer stent 912 using pins, rivets, or other securement devices known in the art. Thus, in the embodiment of FIGS. 10-11, the proximal struts 930 of the inner stent 910 are wider than the distal struts 932 of the inner stent 910, as compared to the distal struts 532 of the inner stent 110 being wider that the proximal struts 530 of the inner stent 110. Similar to the inner stent 110, this results in the inner stent 910 being asymmetric with respect to a lateral axis, that is, an axis perpendicular to the central longitudinal axis CLA of the inner stent 110. Having the proximal struts 930 with a larger width than the distal struts 932 provides increased strength at the location of the connection between the inner stent 910 and the outer stent 912, while maintaining a low packing density by having narrower distal struts 932.

In this embodiment, the outer stent 912 of the frame 908 is inverse of the outer stent 112 described in connection with some embodiments above. Accordingly, the outer stent 912 shown and described herein with respect to FIGS. 11-12 includes cells 930 around a circumference of the outer stent 912. Each cell 930 of the outer stent 912 is defined by a plurality of struts and nodes or crowns. From an inflow or proximal end 925A to an outflow or distal end 925B of the outer stent 912, the outer stent 912 includes first nodes 934A. In the embodiment shown, there are six first nodes 934A around the circumference of the outer stent 912. Extending proximally from the first nodes 934A are eyelets 938, which are configured to align with corresponding eyelets of the inner stent 910 to couple the inner and outer stents 910, 912 together, such as by rivets. A connector arm 936 extends longitudinally from each first node 934A to a corresponding second node 934B. Thus, there are also six second nodes 934B. A first row of first struts 933A extend distally and circumferentially from the first nodes 934A. Distal ends of adjacent first struts 933A are coupled to each other at third nodes 934C. As can be seen, the third nodes 934C do not have any struts or arms extending distally therefrom. Each second node 934B includes two second struts 933B extending distally and circumferentially therefrom. Distal ends of the second struts 933B are coupled to fourth nodes 934D, thereby resulting in twelve fourth nodes 934D around the circumference of the outer stent 912. Extending distally and circumferentially from each fourth node 934D are two third struts 933C. Distal ends of the third struts are coupled to corresponding fifth nodes 934E. Further, the distal ends of adjacent third struts 933C extending from different fourth nodes 934D are coupled to the same fifth node 934E. Extending distally and circumferentially from each fifth node 934E are two fourth struts 933D. The distal ends of the adjacent fourth struts 933D extending from different fifth nodes 934E are coupled at sixth nodes 934F. The sixth nodes 934F define a distal end of the outer stent 912.

As stated previously, the outer stent 912 of the frame 908 includes a plurality of cleats that extend radially outwardly from the outer surface of the outer stent 912 and are configured to engage with the native tissue, further fixating the outer stent 912 to the tissue. In the embodiment shown in FIGS. 11-12, the outer stent 912 includes a first row of first or proximal strut cleats 942, a row of fifth node cleats 944, a row of fourth strut cleats 946 and a row of sixth node cleats 941. Each proximal strut cleat 942 extends from each third strut 933C of the outer stent 912. More specifically, each third strut 933C includes a first side 931A that faces the inflow end 925A of the outer stent 912 when the outer stent 912 is in the radially expanded deployed configuration and a second side 931B that opposes the first side 931A and faces the outflow end 925A of the outer stent 912 when the outer stent 912 is in the radially expanded deployed configuration. Each proximal strut cleat 942 is coupled to the first side 931A of a corresponding third strut 933C of the outer stent 912. The proximal strut cleats 942 may also be referred to as mid-strut cleats as they extend from struts between adjacent nodes. Each fifth node cleat 944 extends from a corresponding fifth node 934D of the outer stent 912. Each fourth strut cleat 946 extends from each fourth strut 933D of the outer stent 912. More specifically, each fourth strut 933D includes a first side 931C that faces the inflow end 925A of the outer stent 912 when the outer stent 912 is in the radially expanded deployed configuration and a second side 931D that opposes the first side 931C and faces the outflow end 925B of the outer stent 912 when the outer stent 912 is in the radially expanded deployed configuration. Each fourth strut cleat 946 is coupled to the first side 931C of a corresponding fourth strut 933D of the outer stent 912. Each sixth node 941 extends from a corresponding sixth node 934E of the outer stent 912. In the embodiment shown, each sixth node cleat 941 may be a forked cleat. The forked sixth node cleats 941 have a substantially “Y-shaped” configuration such that extends from the sixth node 934E and then forks such that the sixth node cleat 941 splits into two cleat ends in a “Y-shaped” configuration. The fifth node cleats 944 and the proximal strut cleats 942 are substantially straight segments that extend from the fifth nodes 934D and the third struts 933C, respectively.

As best shown in FIG. 12, each proximal strut cleat 942, fifth node cleat 944, and sixth node cleat 941 extends proximally and radially outwardly. In other words, the cleats 942, 944, and 941 extend proximally in the y-axis direction and outwardly in the z-axis direction. Further, each fifth node cleat 944 and each sixth node cleat 941 extends substantially perpendicular to the x-axis. In other words, each fifth node cleat 944 and each sixth node cleat 941 is not angled primarily laterally or primarily circumferentially. However, those skilled in the art will recognize that stents do not necessary expand uniformly. Therefore, in the radially expanded deployed configuration, the fifth node cleats 944 and the sixth node cleats 941 may be angled somewhat laterally or circumferentially (i.e., in the x-axis direction) with respect to the central longitudinal axis CLA (i.e., with respect to the y-axis). In embodiments, this angle may be under 10°.

With respect to the x-axis (i.e., lateral axis), each proximal crown cleat 941 and each third node cleat 944 extends substantially straight, i.e. parallel to the CLA of the frame 908. In other words, each distal crown cleat 941 and each third node cleat 944 does not extend at a substantial angle or lean substantially toward either of the fourth struts 933D or second struts 933C that they are disposed between, respectively.

In addition to extending proximally and radially outwardly, each proximal strut cleat 942 extends at an angle from the third strut 933C to which it is coupled. More particularly, each proximal strut cleat 942 extends at an angle in the range of 15°-45°, or 20°-40°, or approximately 30° from the third strut 933C to which it is coupled such that the proximal strut cleat 942 is not parallel to the third strut 933C to which it is coupled, but rather extends from the first strut 933C at an acute angle.

The proximal strut cleats 942 may have a length of about 2 mm-4 mm, the fifth node cleats 944 may have a length of about 2 mm-4 mm, and the sixth node cleats 941 may have a length of about 3 mm-6 mm.

The cleats 942, 944, 941 are shape set to the radially expanded configuration. A tool may be used to move the cleats 942, 944, 941 to the radially expanded configuration prior to shape setting the outer stent 112 to the radially expanded configuration.

Those skilled in the art will recognize that cleats 942, 944, and 941 may be varied from the embodiment described. In particular, the location, angles, and or quantity of the cleats may be varied in keeping with the scope of the present disclosure.

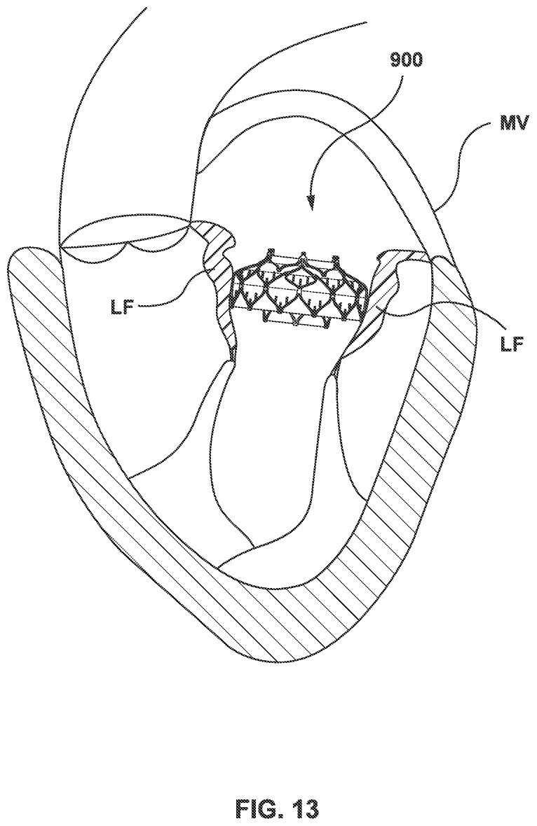

The remaining features of the heart valve prosthesis 900 may be the same as or similar to the heart valve prosthesis 100. Further, although the inner stent 910 of the heart valve prosthesis 900 has been described as similar to the inner stent 110 except being reversed in direction, this is not meant to be limiting, and the inner stent 910 can instead be similar to the inner stent 210, except being reversed in direction. FIG. 13 shows the heart valve prosthesis of FIGS. 11-12 deployed within a native mitral valve, although, as with all previous embodiments described, this embodiment may be deployed within a native tricuspid valve, a native aortic valve, or a native pulmonary valve, for example. These embodiments may also be deployed within previously implanted prosthetic stents, frames, docks, fixation rings, or valves.

As stated previously, the outer diameter of the outer stent 112 of the frame 108 may vary from the inflow end 325A to the transition portion 328 of the outer stent 112. This portion of the outer stent 112 may also be referred to as the fixation region or fixation ring 329 of the outer stent 112. In particular, it may be desirable to vary the shape of the fixation region 329 of the outer stent 112 in order to better match the shape of the native valve in which the transcatheter heart valve prosthesis is to be deployed. The shape of the fixation region 329 may be varied when the outer stent is shape set on a mandrel 1401 by varying the shape of the corresponding portion of the mandrel 1401. FIGS. 14A-14C depicts various embodiments for shape-setting the fixation region 329 of the outer stent 112.

FIG. 14A shows an embodiment wherein the outer diameter of the mandrel 1401 is convex with respect to the central longitudinal axis thereof. Such a shaped mandrel 1401 results in an outer diameter of the fixation region 329 being convex with respect to the central longitudinal axis CLA of the transcatheter heart valve prosthesis. Thus, the outer diameter of the mandrel 1401 and the outer stent 112 curves radially inward from a first diameter at the inflow end 325A to a second diameter smaller than the first diameter approximately mid-way between the inflow end 325A and the transition portion 328, and then curves radially outwardly to a third diameter approximately the same as the first diameter at the transition portion 328.

FIG. 14B shows an embodiment wherein the outer diameter of the mandrel 1401 curves radially outwards adjacent the transition portion 328 of the outer stent 112, resulting in the same shape of the outer stent 112. In particular the outer diameter of the fixation region 329 is approximately constant starting at the inflow end 325A. Adjacent the transition portion 328, the outer diameter of the outer stent 112 curves radially outwardly and then radially back inwardly at the transition portion 328. Accordingly, the portion proximal adjacent the inflow end 325A has a smaller outer diameter than the portion adjacent the transition portion 328.

FIG. 14C shows an embodiment wherein the outer diameter of the mandrel 1401, and hence the outer diameter of the outer stent 112 is concave with respect to the central longitudinal axis CLA. In particular, the outer diameter of the fixation region 329 curves radially outwardly from a first diameter at the inflow end 325A to a second diameter larger than the first diameter approximately mid-way between the inflow end 325A and the transition portion 328, and then curves back to a third diameter approximately equal to the first diameter at the transition portion.

It should be understood that these variations can also be applied to the outer stent 912 described above.

As stated previously, the outer stent 112 can include T-bar connectors 326 that enable the outer stent 112 to be coupled or connected to a delivery system. More specifically, as shown in FIGS. 14A-14C, every other distal crown aperture of the outer stent 112 includes a T-bar connector 326 that extends distally therefrom and that may be used to connect the prosthesis to a delivery system for delivering the prosthesis into a patient's body. Thus, the outer stent 112 includes three T-bar connectors 326. The T-bar connectors 326 of the outer stent 112 enable the outer stent 112 to be coupled or connected to a delivery system. However, this is not meant to be limiting, as there may be more or fewer T-bar connectors 326. The T-bar connectors 326 shown in FIGS. 14A-14C are T-shaped, but such connectors may be shapes other than T shaped, such as L-shaped or C-shaped as known to those skilled in the art, as long as the shape is capable of functioning as a connector.

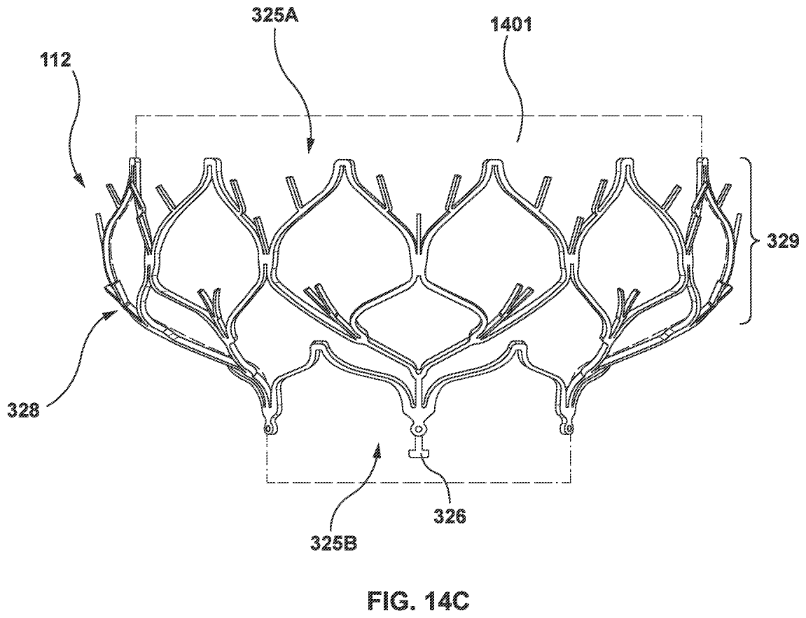

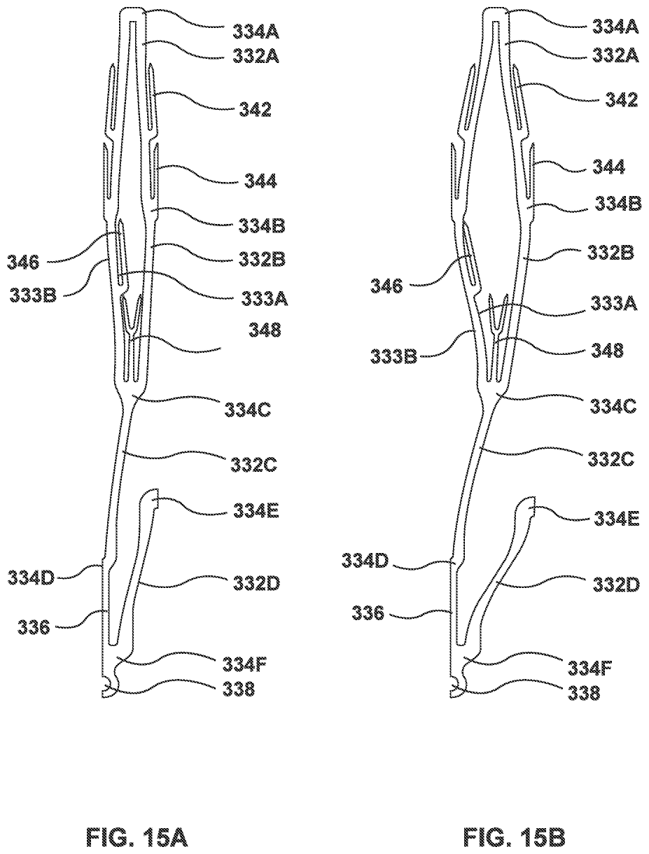

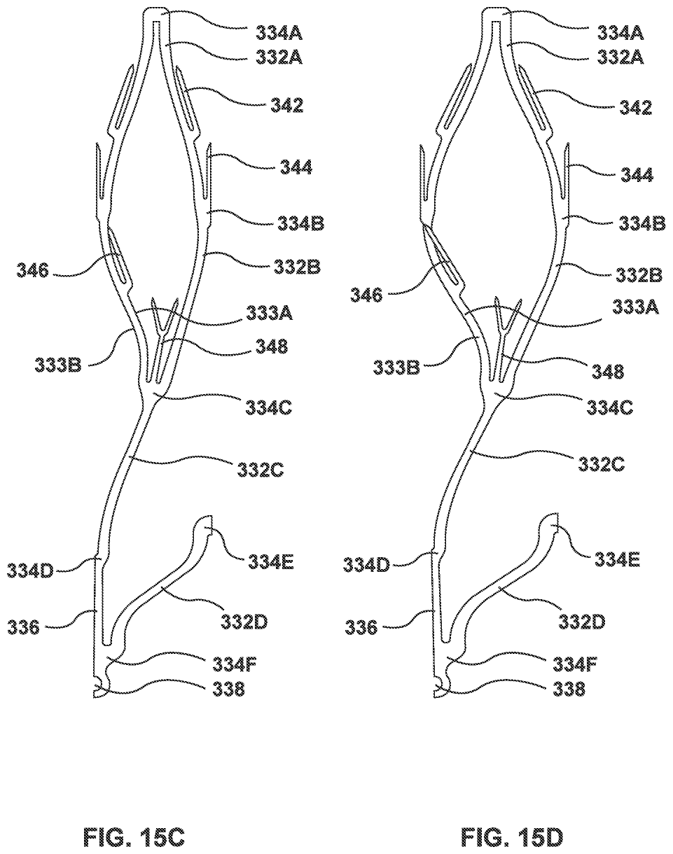

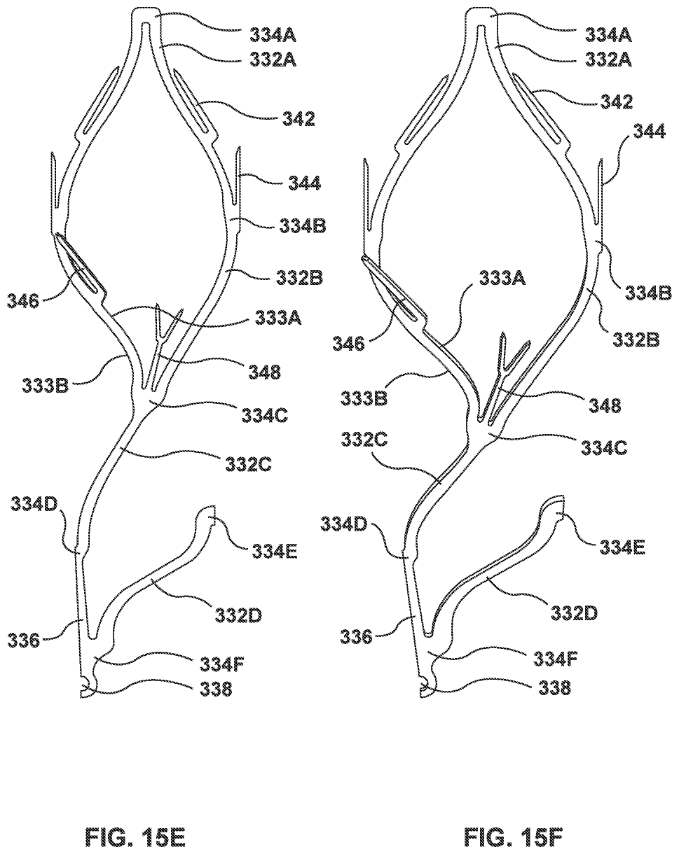

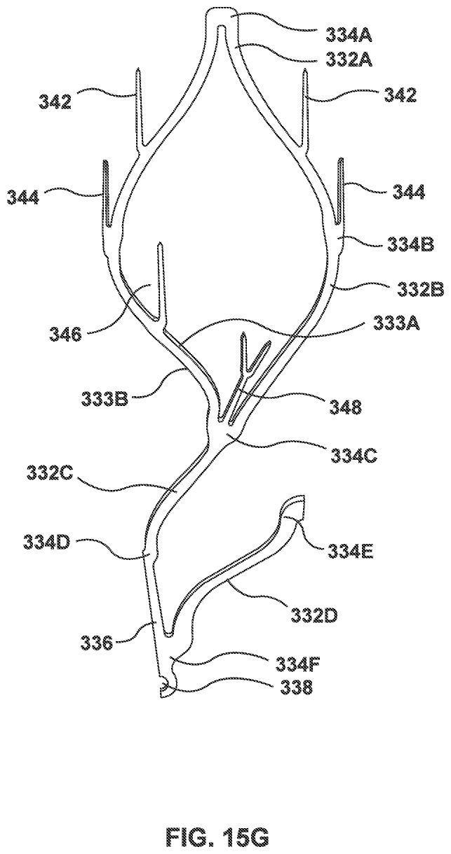

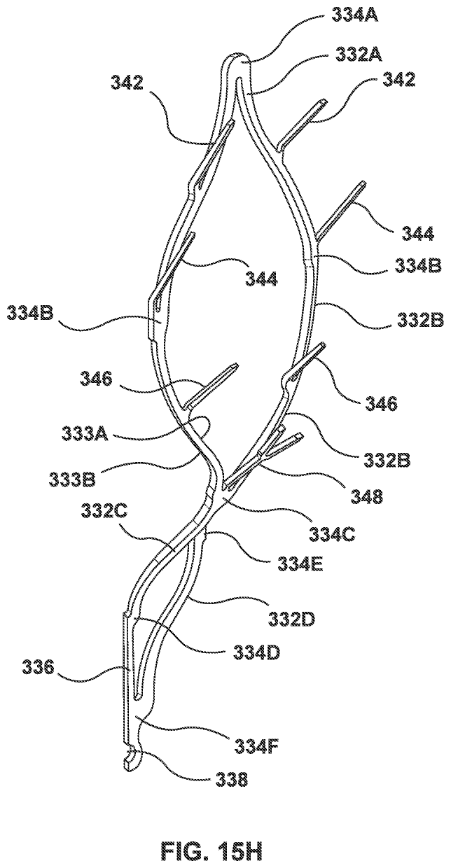

FIGS. 15A-15F show a portion of the outer stent 112 according to another embodiment hereof as it radially expands from the reduced diameter configuration of FIG. 15A to the radially expanded deployed configuration of FIG. 15F. Although FIGS. 15A-15F show only a portion of the outer stent 112, those skilled in the art would recognize that the pattern shown repeats itself around the circumference of the outer stent, as shown in FIGS. 1-3 above. In the embodiment shown in FIGS. 15A-15F, in addition to the cleats described with respect to FIGS. 1-3, the outer stent 112 further includes a second strut cleat 346 coupled to every other second strut 332B. More specifically, each second strut 332B includes a first side 333A that faces the inflow end 325A of the outer stent 112 when the outer stent 112 is in the deployed configuration, as shown in FIG. 15F, and a second side 333B that faces the outflow end 325B of the outer stent 112 when the outer stent 112 is in the deployed configuration. Each second strut cleat 346 is coupled to every other first strut 332B on the first side 333A of the second strut 332B, as shown in FIGS. 15A-15F.

As can be seen, FIG. 15A depicts the portion of the outer stent 112 in the compressed or delivery configuration and FIG. 15G depicts the portion of the outer stent 112 in the radially expanded deployed configuration. FIGS. 15B-15H show the transition of the outer stent 112 from the delivery configuration to the deployed configuration. As the outer stent 112 transitions to the deployed configuration, the cells 330 of the outer stent 112 expand such that the space between the pair of adjacent second struts 332B and the space between the pair of first struts 332A increases. As described above, the cleats, including the second strut cleat 346, is shape set to the radially expanded configuration. A tool may be used to push the cleats, including the second strut cleat 346, to the radially expanded configuration prior to shape setting the outer stent 112, as shown in FIG. 15G. As can be seen in the perspective view of FIG. 15H, the proximal strut cleats 342, second node cleats 344 and second strut cleat 346 are splayed out such that each cleat 342, 344, 346 extends proximally with respect to the central longitudinal axis CLA and radially outwardly with respect to the z-axis of the outer stent 112. The proximal strut cleats 342, second node cleats 344 and second strut cleats 346 do not extend circumferentially or laterally with respect to the x-axis of the outer stent 112. The forked cleat 348 extends proximally with respect to the CLA, radially outwardly with respect to the z-axis, and slightly extends laterally with respect to the x-axis of the outer stent 112. It should be understood that the embodiment shown and described with respect to FIGS. 15A-15F can be applied to both the outer stent 112 and the outer stent 912 described above.

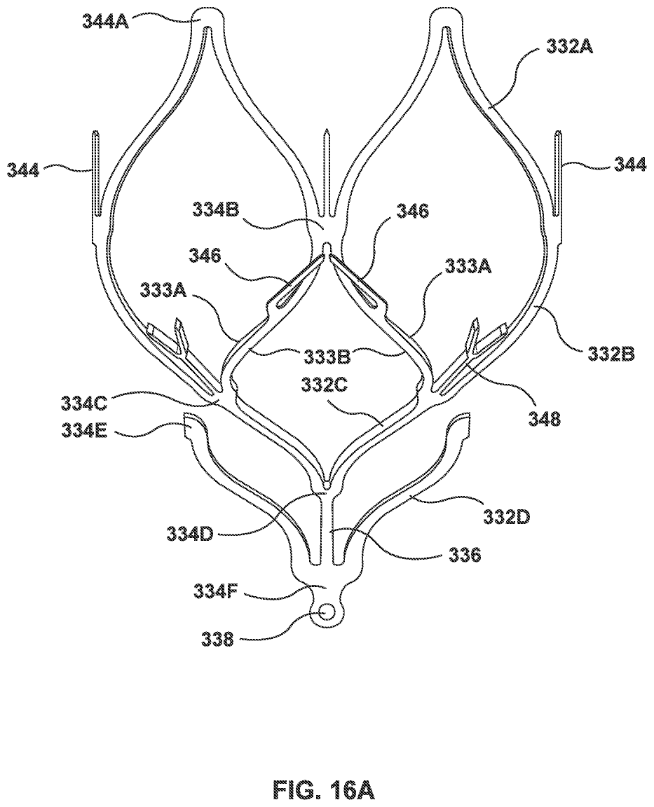

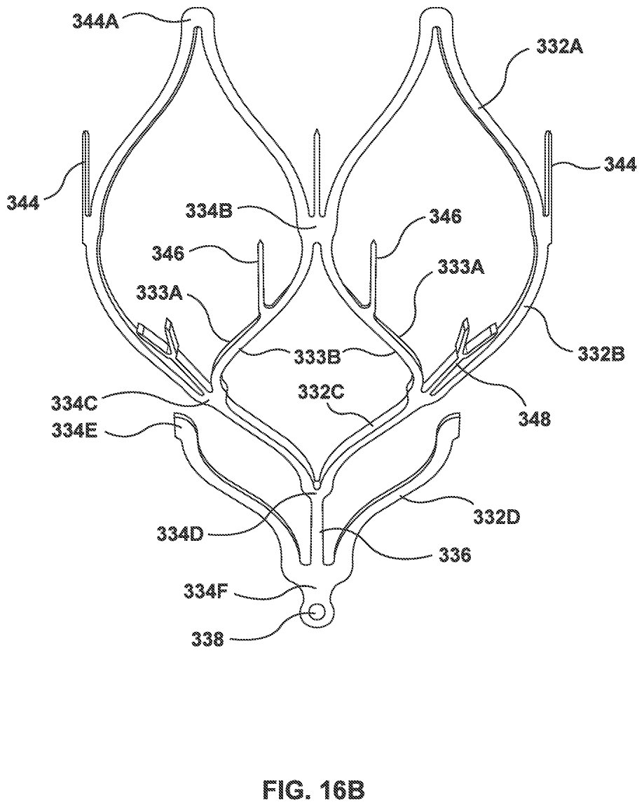

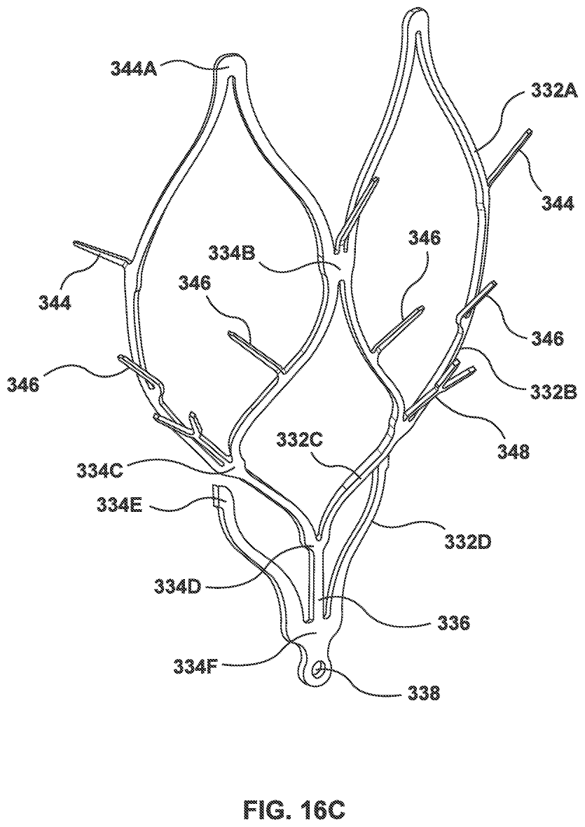

FIG. 16A shows a portion of the outer stent 112 according to another embodiment hereof. In the embodiment shown, the outer stent 112 includes a pair of second strut cleats 346 that are coupled to every other pair of two adjacent second struts 332B. As shown in FIG. 16A, one of the second strut cleats 346 is coupled to a first side 333A of a second strut 332B and the other second strut cleat 346 is coupled to the first side 333A of the adjacent second strut 332B. In the embodiment of FIG. 16A, the adjacent second struts 332B with the second strut cleats 346 are coupled to a common one of the second nodes 334B. Further, the second struts 334B extending distally from each of the second nodes 334B adjacent to the second node 334B that includes the second struts 334B with the second strut cleats 336 do not include strut cleats. The second strut cleats 346 are shown overlapping the second struts 332B to which they are coupled, however, FIG. 16A is prior to shape setting the outer stent 112 such that the second strut cleats 346 extend radially outwardly, proximally, and away from the first side 333A of the second strut 332B, as shown in FIG. 16B. As mentioned above, a tool may be used to push the cleats, including the second strut cleats 346, to the radially expanded configuration prior to shape setting the outer stent 112. As can be seen in FIG. 16C, the second node cleats 344 and second strut cleats 346 are also shape set to be splayed out such that each cleat 344, 346 extends proximally with respect to the central longitudinal axis CLA and radially outwardly with respect to the z-axis of the outer stent 112 but does not extend substantially laterally with respect to the x-axis of the outer stent 112. Each forked cleat 348 extends proximally with respect to the CLA, radially outwardly with respect to the z-axis, and extends slightly in the lateral direction with respect to the x-axis of the outer stent 112. It should be understood that the embodiment shown and described with respect to FIGS. 16A-16C can be applied to both the outer stent 112 and the outer stent 912 described above.

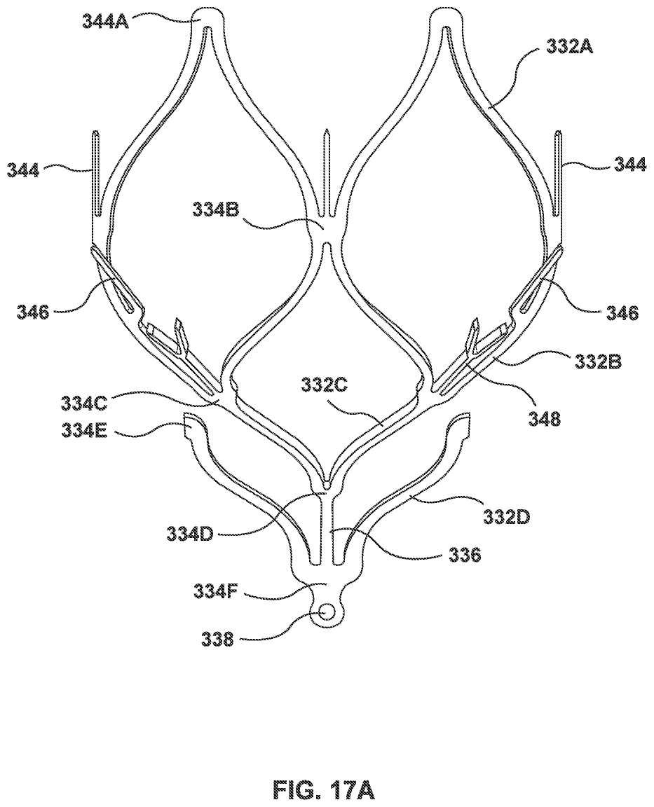

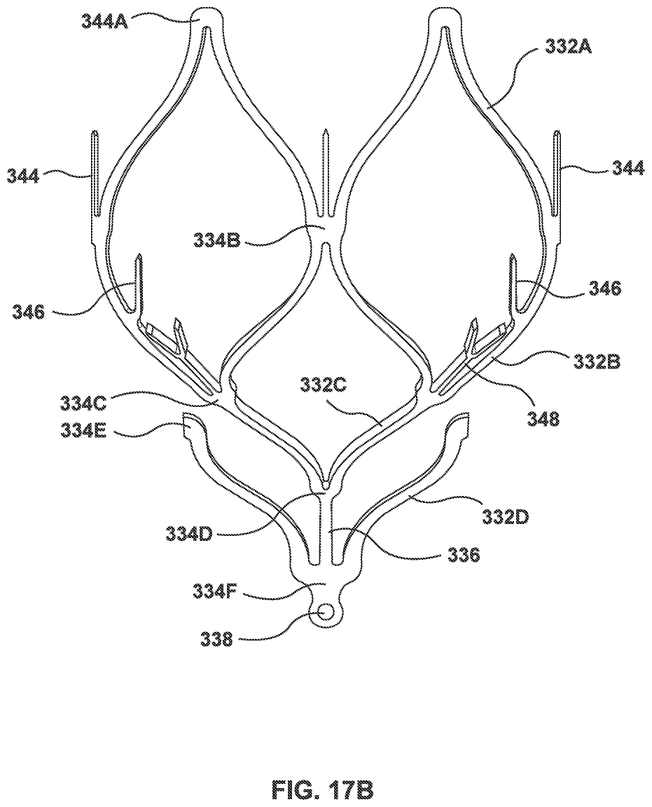

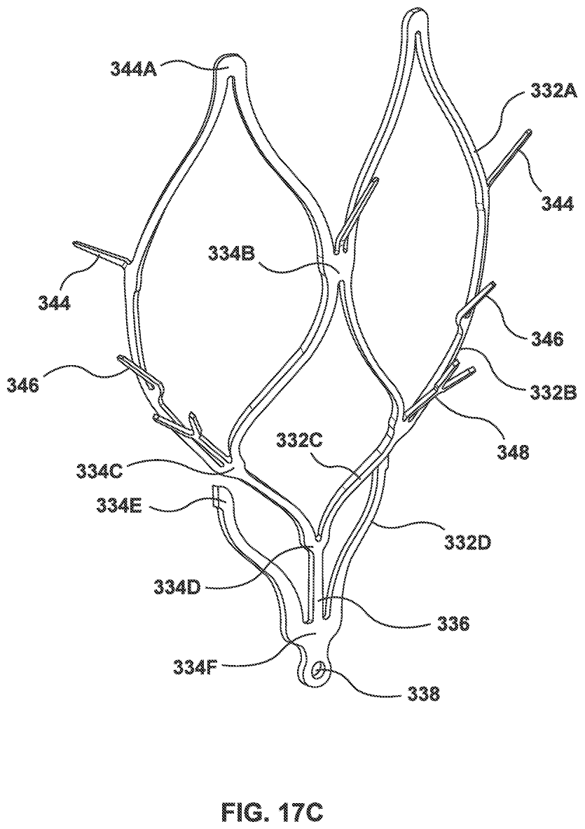

FIG. 17A shows a portion of the outer stent 112 according to another embodiment hereof. As can be seen, between each pair of second struts 332B that have second strut cleats 346 coupled thereto are a pair of first struts 332B that do not have strut cleats coupled thereto. The second strut cleats 346 are shown overlapping the second struts 332B to which they are coupled, however, FIG. 17A is prior to shape setting such that the second strut cleats 346 extend radially outwardly, proximally, and away from the second strut 332B, as shown in FIG. 17B. As mentioned above, a tool may be used to push the cleats, including the second strut cleat 346, to the radially expanded configuration prior to shape setting the outer stent 112. As can be seen in FIG. 17C, the second node cleats 344 and second strut cleats 346 are shape set to be splayed out such that each cleat 344, 346 extends proximally with respect to the central longitudinal axis CLA and radially outwardly with respect to the z-axis of the outer stent 112 but does not extend substantially laterally with respect to the x-axis of the outer stent 112. Each forked cleat 348 extends proximally with respect to the CLA, radially outwardly with respect to the z-axis, and extends slightly in the lateral direction with respect to the x-axis of the outer stent 112. It should be understood that the embodiment shown and described with respect to FIGS. 17A-17C can be applied to both the outer stent 112 and the outer stent 912 described above.

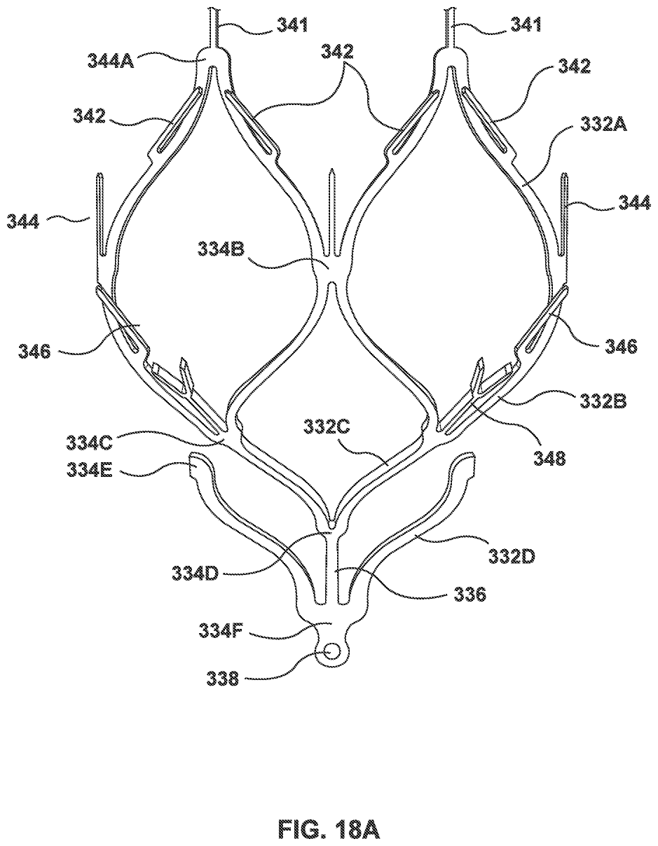

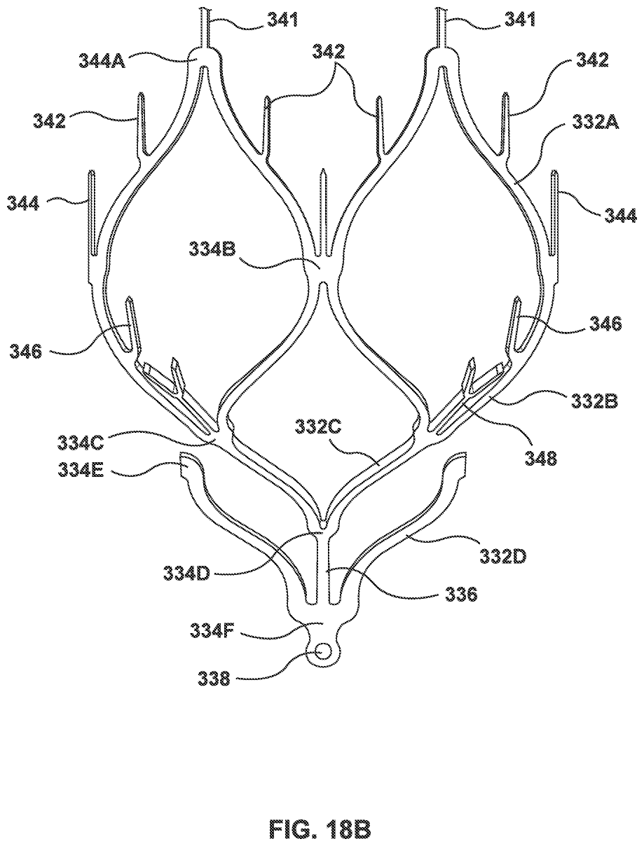

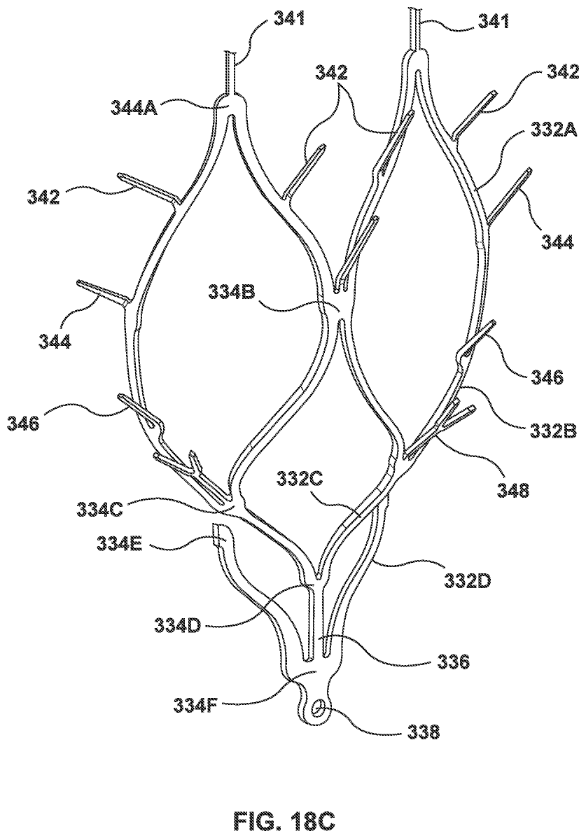

FIG. 18A shows a portion of the outer stent 112 according to another embodiment hereof. The embodiment shown in FIG. 18A is the same as the outer stent 112 shown in FIGS. 16A-17B, except that the embodiment shown in FIG. 18A includes the proximal strut cleats 342 as shown in FIGS. 1-3 and also includes a first node cleat 341 coupled to each first node 334A of the outer stent 112. As shown, each first node cleat 341 is coupled to and extends proximally from each proximal crown 334A of the outer stent 112. Each first node cleat 341 extends proximally with respect to the y-axis (CLA) and radially outwardly with respect to the z-axis. In this embodiment, the outer stent 112 also includes a pair of second strut cleats 346 on every other pair of second struts 332B, similar to the outer stent 112 shown in FIGS. 16A-17B. As mentioned above, a tool may be used to push the cleats, including the proximal strut cleats 342 and the second strut cleats 346, to the radially expanded configuration prior to shape setting the outer stent 112. As can be seen in FIG. 18C, the first node cleats 341, proximal strut cleats 342, second node cleats 344 and second strut cleats 346 are shape set to be splayed out such that each cleat 341, 342, 344, 346 extends proximally with respect to the central longitudinal axis CLA and radially outwardly with respect to the z-axis of the outer stent 112 but does not extend substantially laterally with respect to the x-axis of the outer stent 112. Each forked cleat 348 extends proximally with respect to the CLA, radially outwardly with respect to the z-axis, and extends slightly in the lateral direction with respect to the x-axis of the outer stent 112. It should be understood that the embodiment shown and described with respect to FIGS. 18A-18C can be applied to both the outer stent 112 and the outer stent 912 described above.

While various embodiments according to the present invention have been described above, it should be understood that they have been presented by way of illustration and example only, and not limitation. It will be apparent to person skilled in the relevant art that various changes in form and detail can be made therein without departing from the spirit and scope of the invention. Thus, the breadth and scope of the present invention should not be limited by any one of each embodiment discussed herein, and of each reference cited herein, can be used in combination with the features of any other embodiment. All patents and publication discussed herein are incorporated by reference herein in their entirety.

1. A heart valve prosthesis having first and second ends, and configured to be radially compressed to a radially compressed configuration and radially expanded to a radially expanded configuration, the heart valve prosthesis comprising:
an inner stent:
an outer stent surrounding at least a portion of the inner stent and operatively coupled to the inner stent, and configured to secure the heart valve prosthesis to native heart tissue, the outer stent including:
a plurality of struts and a plurality of nodes defining open cells of the outer stent, wherein the plurality of struts are substantially straight segments and each of the plurality of nodes connects adjacent struts of the plurality of struts, and
a plurality of cleats, the plurality of cleats including first strut cleats extending radially outwardly and toward the first end of the prosthesis from first struts of a first row of first struts of the plurality of struts and between adjacent nodes in the radially expanded configuration:
and a prosthetic valve operatively coupled to the inner stent.

2. The heart valve prosthesis of claim 1, wherein the plurality of nodes includes a first row of first nodes around a circumference of the outer stent at a first end of the outer stent and a second row of second nodes that is closer to the second end of the heart valve prosthesis than the first row of first nodes is to the second end, and wherein the first struts of the first row of first struts have a first end coupled to a corresponding first node and a second end coupled to a corresponding second node.

3. The heart valve prosthesis of claim 2, wherein the plurality of cleats further includes a plurality of second node cleats extending radially outwardly and from corresponding second nodes of the second row of second nodes and in the direction of the first end of the heart valve prosthesis in the radially expanded configuration.

4. The heart valve prosthesis of claim 2, wherein the plurality of nodes further includes a third row of third nodes around a circumference of the outer stent that is closer to the second end of the heart valve prosthesis than the second row of second nodes is to the second end of the heart valve prosthesis, and wherein the plurality of struts includes a second row of second struts with each second strut having a first end coupled to a corresponding second node and a second end coupled to a corresponding third node.

5. The heart valve prosthesis of claim 4, wherein the plurality of cleats further includes a plurality of third node cleats extending radially outwardly from corresponding third nodes of the third row of third nodes and in the direction of the first end of the heart valve prosthesis in the radially expanded configuration.

6. The heart valve prosthesis of claim 5, wherein the third node cleats are forked cleats.

7. The heart valve prosthesis of claim 4, wherein the outer stent further includes a plurality of second strut cleats extending radially outwardly from the second struts and in the direction of the first end of the heart valve prosthesis in the radially expanded configuration.

8. The heart valve prosthesis of claim 2, wherein the plurality of cleats further includes a plurality of first node cleats extending radially outwardly from corresponding first nodes of the first row of first nodes and in the direction of the first end of the heart valve prosthesis in the radially expanded configuration.

9. The heart valve prosthesis of claim 1, wherein the outer stent and the inner stent are operatively coupled to each other at outflow ends thereof.

10. The heart valve prosthesis of claim 1, wherein the outer stent and the inner stent are operatively coupled to each other at inflow ends thereof.

11. The heart valve prosthesis of claim 1, wherein the outer stent includes a first end coupled to the inner stent, a transition region flaring radially outwardly from the first end, and a fixation region extending from the transition region to a second end of the outer stent.

12. The heart valve prosthesis of claim 11, wherein the fixation region is concave with respect to a central longitudinal axis of the transcatheter heart valve prosthesis.

13. The heart valve prosthesis of claim 11, wherein the fixation region is convex with respect to a central longitudinal axis of the transcatheter heart valve prosthesis.

14. The heart valve prosthesis of claim 11, wherein an outer diameter of the fixation region curves radially outwardly adjacent a junction of the fixation region and the transition region.

15. The heart valve prosthesis of claim 11, wherein the first end is an inflow end of the inner stent.

16. The heart valve prosthesis of claim 11, wherein the first end is an outflow end of the inner stent.

17. The heart valve prosthesis of claim 1, wherein the first strut cleats extend from a portion of the first struts that is between a first node and a second node at respective first and second ends of the first struts.

18. The heart valve prosthesis of claim 1, wherein the first strut cleats extend from the first struts at or around the midpoint on the first struts and between a first node and a second node at respective first and second ends of the first struts.

19. (canceled)

20. (canceled)

21. The heart valve prosthesis of claim 1, wherein the inner stent includes:
a plurality of inner stent cells positioned around a central longitudinal axis of the heart valve prosthesis, each inner stent cell being defined by:
two inner first struts, each inner first strut having a first width;
a first crown joining first ends of the inner first struts;
two inner second struts, each inner second strut having a second width that is larger than the first width;
a second crown joining second ends of the inner second struts; and
two connectors, each connector coupling a second end of a respective one of the two inner first struts to a first end of a respective one of the two inner second struts;

22. The heart valve prosthesis of claim 1, wherein the inner stent includes:
a plurality of inner stent cells positioned around a central longitudinal axis of the heart valve prosthesis, each inner stent cell being defined by:
two inner first struts;
a first crown joining first ends of the inner first struts;
two inner second struts;
a second crown joining second ends of the inner second struts; and
two connectors, each connector coupling a second end of a respective one of the two inner first struts to a first end of a respective one of the two inner second struts;
a plurality of inner support struts including:
a first inner support strut having a second end thereof coupled to the second end of a first second strut of the inner second struts and/or the second crown of the first second strut;
a second inner support strut having a second end thereof coupled to the second end of a second second strut of the inner second struts circumferentially adjacent to the first second strut and/or the second crown of the second second strut; and
a support crown joining first ends of the first inner support strut and the second inner support strut,
wherein the inner support struts are arranged between circumferentially adjacent second crowns of the inner stent in an inverted V-shaped configuration.
